(19) United States
(12) Patent Application Publication
(10) Pub. No.: US 2008/0199514 A1
(43) Pub. Date: Aug. 21, 2008

(54) Functional Variant of Lymphoid Tyrosine Phosphatase is Associated with Autoimmune Disorders

(75) Inventors: Tomas Mikael Mustelin, San Diego, CA (US); Nunzio Bottini, Pasadena, CA (US)

Correspondence Address: CATALYST LAW GROUP, APC, 9710 SCRANTON ROAD, SUITE S-170, SAN DIEGO, CA, 92121, US

(73) Assignee: THE BURNHAM INSTITUTE, La Jolla, CA (US)

(21) Appl. No.: 11/571,711

(22) PCT Filed: Jul. 11, 2005
(86) PCT No.: PCT/US05/24862

§ 371 Date: Apr. 19, 2007

Related U.S. Application Data
(60) Provisional application No. 60/586,633, filed on Jul. 9, 2004.

(51) Int. Cl. C12Q 1/42; C12Q 1/68; G01N 33/50; A61K 31/70; A61K 39/395; C12N 9/16; C12N 15/52; A61K 31/7088; A61K 9/127; A61K 38/02; C12Q 1/02

(52) U.S. Cl. 424/450; 435/6; 435/29; 435/21; 435/7.1; 435/7.92; 514/2; 514/23; 424/130.1; 514/44; 435/196; 536/23.2

(57) ABSTRACT

The invention is the discovery of a single nucleotide polymorphism in the gene coding for lymphoid tyrosine phosphatase. This SNP leads to a mutation in the lymphoid tyrosine phosphatase protein that prohibits binding with the SH3 domain of Csk, and leads to a subsequent dysregulation of the T-Cell activation cascade. Such dysregulation can lead to a variety of autoimmune disorders. Thus, the invention provides a series of useful methods for diagnosing, discovering modulators, developing treatments and providing research tools. Also, the invention provides compositions of matter useful for research and useful for the diagnosis and treatment of these disorders.

### RELATED APPLICATION

Benefit of priority under 35 U.S.C. 119(e) is claimed herein to U.S. Provisional Application No.: 60/586,633, filed Jul. 9, 2004. The disclosure of the above referenced application is incorporated by reference in its entirety herein.

### STATEMENT ON FEDERALLY SPONSORED RESEARCH

This invention was made in part with United States government support under grant number NIH AI053585 awarded by the National Institutes of Health. The United States government has certain rights in this invention.

### FIELD OF THE INVENTION

This invention relates generally to lymphoid tyrosine phosphatase mediated T-Cell Activation and autoimmune disorders, and more specifically relates to the discovery of a single nucleotide polymorphism in the lymphoid tyrosine phosphatase gene resulting in a lymphoid tyrosine phosphatase protein having a single amino acid mutation at residue 620.

### BACKGROUND

B and T lymphocytes are the central mediators of humoral and cellular immune responses to pathogenic and “non-self” antigenic challenges. The B and T cell antigenic receptors, BCR and TCR, respectively, are the focal points for signals that drive B and T lymphocyte-specific differentiation, dictate proliferative programs as well as trigger a full immune response or programmed cell death. The immune response is rapid and tightly controlled; this is achieved by regulation of BCR and TCR signaling complexes that include a multiplicity of associated kinases and adaptor proteins.

Lineage specific receptor and cytoplasmic protein tyrosine kinases (PTKs), such as those of the SRC family (Lyn, Lck, Fyn), Btk/Tec family (Btk, Etk) and Syk family (Syk, ZAP-70) are activated upon receptor activation, leading to the phosphorylation and recruitment of adaptor molecules via immunoreceptor tyrosine-based activation and inhibition motifs and SH2 binding motifs. Src activity is regulated by tyrosine phosphorylation at two sites with opposing effects. Phosphorylation of Tyr416 in the activation loop of the kinase domain upregulates enzyme activity. Phosphorylation of Tyr527 in the carboxy-terminal tail renders the enzyme less active. Similarly, the Src family proteins Lck and Fyn are regulated by Csk phosphorylation. For example, Lck, which is essential for T-lymphocyte activation and differentiation, is phosphorylated at Tyr505 in the carboxy terminal to downregulate its catalytic activity, while phosphorylation at Tyr394 increases activity. Removal of a phosphate at Tyr 494 of Lyk further downregulates the enzyme's catalytic activity

An essential and important function of the SRC pathways is a strict negative regulation of lymphocyte function to prevent autoimmune and lymphoproliferative pathologies. Negative regulation is mediated by c-terminal Src kinase (Csk) protein and lipid phosphatases, as well as c-Cbl ubiquitin ligase. For example, Lck is essential for T-lymphocyte activation and differentiation. Phosphorylation of Tyr505 and dephosphorylation of Tyr494 of Lck downregulates its catalytic activity, while phosphorylation of Tyr394 leads to an increase in Lck activity.

Protein Tyrosine Phosphatases (PTPs) are important regulators of the immune response and are involved in maintaining the resting phenotype of lymphocytes as well as in the control of signaling from antigen receptors, co-stimulatory receptors, and cytokine receptors. The lymphoid-specific phosphatase (LYP) encoded by the PTPN22 gene is a 110-kDa PTP consisting of an N-terminal phosphatase domain and a long non-catalytic C-terminus with several proline-rich motifs. LYP is expressed in lymphocytes, where it physically associates through its most N-terminal proline-rich motif (termed P1) with the SH3 domain of the Csk kinase, an important suppressor of the Src family kinases Lck and Fyn, which mediate T cell antigen receptor signaling. LYP is among the most powerful inhibitors of T cell activation, a task accomplished by dephosphorylation of TCR-associated kinases, like Lck, Fyn, and ZAP-70. Dephosphorylation of ZAP-70 may be connected to the negative regulatory function of the c-Cbl proto-oncogene, since LYP forms a complex with c-Cbl.

Single-nucleotide polymorphisms (SNPs) are common in the human genome and often provide correlative evidence for the involvement of specific genes in human disease. SNPs that impact the function of critical components of the T cell antigen receptor (TCR) signaling pathways could have profound effects on the function of the immune system and thus the development of autoimmune diseases. Protein tyrosine phosphatases (PTPs) are particularly attractive candidates for harboring disease-related SNPs because they are involved in preventing spontaneous T cell activation and they restrict the response to antigen by dephosphorylating and inactivating TCR-associated kinases and their substrates. PTPases are also needed for the reversion of activated T blasts to a resting phenotype.

### SUMMARY OF THE INVENTION

One embodiment of the present invention relates to the discovery of a single nucleotide polymorphism in the gene coding for lymphoid tyrosine phosphatase. This SNP causes a variation in amino acid sequence for the lymphoid tyrosine phosphatase protein, which subsequently leads to a variety of autoimmune disorders.

In one particular aspect of the present invention, this discovery leads to materials and methods useful for research, diagnosis and treatment of disorders relating to the SNP mutation in lymphoid tyrosine phosphatase.

### BRIEF DESCRIPTION OF THE DRAWINGS

FIG. 1 shows the amino acid sequence of the two lymphoid tyrosine phosphatase alleles, with the P1 motif indicated.

FIG. 2 illustrates the structure of the P1 motif of lymphoid tyrosine phosphatase bound to the SH3 domain of Csk.

FIG. 3 illustrates the electron clouds of the two residues during interaction of R620 of LYP with W47 of the SH3 of Csk.

FIG. 4 shows binding of an S-tagged LYP polypeptide corresponding to amino acid residues 603-710 with arginine (*C1858 allele) or tryptophan (*T1858 allele) at position 620 to GST (lanes 1 and 2) or GST-Csk-SH3 (lanes 3 and 4). The bound material was detected with S-protein-horse radish peroxidase (HRP). Both forms of the peptide were equally expressed in E. coli.

FIG. 5 shows anti-HA immunoblot (top panel) of material precipitated with GST-Csk-SH3 from COS cells transfected with three different amounts of LYP expression plasmid with either arginine (R) or tryptophan (W) at position 620. Anti-GST blot of the same filter (middle panel) and anti-HA blot of lysates from the same transfectants (bottom panel) to show equal expression of the two allelic forms of LYP.

FIG. 6 shows anti-GST immunoblot (top panel) of material immunoprecipitated with LYP from COS cells transfected with GST-Csk and the two LYP proteins. Anti-HA blot (middle panel) and anti-GST blot (bottom panel) of lysates from the same transfectants to show equal expression.

FIG. 7 shows anti-Csk immunoblot (upper panel) of endogenous Csk precipitated with the S-tagged LYP(603-710) protein with either arginine (R) or tryptophan (W) at position 620 from Jurkat T cells. Lane 6 is material bound to S-agarose alone, lane 7 is total lysates of Jurkat cells, and lanes 8 and 9 are the S-tagged protein alone. S-protein-HRP blot of the same samples (lower panel).

FIG. 8 shows the results from RFLP-PCR-based genotyping assay. Shown are two representative individuals of each of the three genotypes *C*C, *C*T, and *T*T.

FIG. 9 shows the results of an example sequencing reaction of PTPN22 from a heterozygous individual. The arrow indicates the presence of both T and C at position 1858 in codon 620.

FIG. 10a shows the relative phosphatase activity of the mutant LYP W620 as compared with wild-type LYP having an arginine residue at position 620.

FIG. 10b shows a western blot showing immunoprecipitates of co-expressed Zap-70 with Csk and one of three different amounts of hemagglutinin tagged LYP R620, hemagglutinin-tagged LYP W620, or pEF-HA vector alone; in the upper and middle panels the immunoprecipitates are blotted with anti-Zap70 and anti-pTyr; in the lower panel, the blot is anti-HA.

FIG. 11 shows a western blot of Flag Tagged LYP R620 expressed from a recombinant baculovirus system.

FIG. 12. Reduced interleukin-2 production of primary T lymphocytes from T1D patients of the WR genotype compared to patients of RR genotype. IRB approval for the study was obtained at the University of Sassari, Italy, and informed consent forms were signed by each patient or parent. T lymphocytes were isolated from venous blood from T1D patients by Ficoll gradient centrifugation and further purified by immunodepletion of non-T cells. Cells were stimulated with anti-CD3 and anti-CD28 mAb-coated beads (Dynal Inc., Norway) for 20 h at the indicated bead/cell ratio, or with 40 nM phorbol 12-myristate, 13-acetate plus 10 μM ionomycin, or medium alone. Interleukin-2 in the culture supernatant was measured in triplicate by ELISA (Quantikine™, R. & D. Systems Inc., Minneapolis, Minn.). Graph represents mean ±SEM of data from 4 RR patients (black bars) and 5 RW patients (white bars). The statistical significance of the differences between RW and RR was calculated by Student's t-test. n.s., not significant.

FIG. 13. The disease-associated LYP*W620 inhibits T cell activation more potently than LYP*R620. a, Interleukin-2 secretion by primary human T lymphocytes transfected by nucleofection (Amaxa Inc., Germany) with empty vector or HA-tagged LYP*R620 or LYP*W620 and then stimulated for 20 h with anti-CD3ε and anti-CD28 mAbs coated beads (Dynal, Norway), bead to cell ratio 1:1. Interleukin-2 in the culture medium was measured by ELISA (Quantikine™, R. & D. Systems Inc., Minneapolis, Minn.). Data represent mean ±SD from triplicate cell cultures in one of three independent experiments with similar results. b, expression of LYP proteins in the same transfectants as in a (upper panel) and anti-LAT blot as loading control of the same filter (lower panel). c, activation of the NFAT/AP-1-luciferase reporter in primary human T lymphocytes transfected by nucleofection with 1 μg or 3 μg of LYP expression plasmid and then stimulated as in panel a for 6 h, as before. The graph represents the ratio between firefly and renilla luciferase activity and shows mean ±SD from triplicate determinations in one of two independent experiments with similar results. d, expression of the LYP proteins in the same samples as in c (upper panel) and anti-LAT blot as loading control of the same filter (lower panel). e, similar NFAT/AP-1-luciferase assay in Jurkat cells transfected by electroporation with a range of doses of LYP*W620 (filled circles) or LYP*R620 (open circles) expression plasmids and then stimulated with the optimal concentration (150 ng/ml) of anti-CD3ε mAb for 6 h. The data were normalized for LYP expression (arbitrary units) and are given as mean ±SD from triplicate determinations in one of five independent experiments with similar results. When error bars are not seen, they are within the resolution of the data points; lines are nonlinear fits of experimental data.

FIG. 14. The disease-associated LYP*W620 is a more potent inhibitor of early TCR signaling and a more active PTP. a, Phosphorylation of Lck at its positive regulatory site, Y394 (upper panel) in primary T lymphocytes nucleofected with LYP*W620 or LYP*R620 and activated with anti-CD3ε mAb and F(ab)2 fragments of anti-mouse Ig for 0, 2, or 5 min. Control blot for total Lck (middle panel) and anti-HA blot for LYP expression (left bottom panel), and anti-LAT blot (right bottom panel) as loading control for the LYP filter. Similar results were obtained in three independent experiments. b, Tyrosine phosphorylation of TCRζ in primary T lymphocytes nucleofected with LYP*W620 or LYP*R620 and activated as in panel a (upper panel). Control blot for total TCRζ (middle panel), anti-HA blot for LYP expression (left bottom panel), and anti-LAT blot (right bottom panel) as loading control for the LYP blot. Data shown are representative of four independent experiments. c, Calcium mobilization in response to anti-CD3ε mAb in Jurkat T cells co-transfected with LYP and GFP. Cells were loaded with indo-1, as described, and indo-1 fluorescence followed in several thousand GFP+cells. Graph shows data from vector control cells (red), and cells expressing LYP*R620 (blue), or LYP*W620 (green). A control anti-HA blot of the same cells show equal LYP expression. d, Dephosphorylation of an Lck phosphopeptide (ARLIEDNEpYTAAREG) by immunoprecipitated HA-tagged LYP*W620 and LYP*R620 in 100 mM Bis/Tris, pH 6.0, 150 mM NaCl, 1 mM dithiotreitol. Nonenzymatic hydrolysis of the peptide was corrected by measuring the control with addition of heat-inactivated (100° C., 10 min) immunoprecipitates. Another set of controls were obtained by adding 5 mM sodium orthovanadate to the assays, and gave comparable results. The graph represents LYP*W620 activity relative to the activity of LYP*R620 and shows the mean ±SEM from five independent experiments. Statistical analysis on the absorbance data normalized for LYP expression (arbitrary units) showed that differences in the activity between LYP*W620 and LYP*R620 were significant in each one of the five experiments (paired t-test, df=2-4, two-tailed p values were between 0.000025 and 0.019). e, Anti-HA (upper panel) and anti-Csk (lower panel) blots of a representative anti-HA immunoprecipitate used in d.

### DETAILED DESCRIPTION OF THE INVENTION

### Definitions Used Herein

The term “peptide” and the term “polypeptide” are used interchangeably herein.

The term “a peptide similar to . . . ” refers to a peptide having preferably 50% sequence identity with the referenced peptide, more preferably having 75% sequence identity with the referenced peptide, even more preferably having 85% sequence identity with the referenced peptide; and most preferably having 99% sequence identity with the referenced peptide. Sequence identity is preferably determined using BLAST and more preferably using CLUSTAL W; however, those of skill in the art will readily determine sequence identity using a variety of algorithms. (See, e.g.; Baxevanis, A. D., and Ouellette, B. F. F., Bioinformatics, 2 Ed., John Wiley & Sons, Inc., (2001)).

The term “substantially purified” refers to a macromolecule or small molecule that exists in a state where its specific activity, as measured by the activity per physical unit of macromolecule or small molecule present in the preparation, is significantly greater than occurs in vivo, typically at least 10 times greater, more typically at least 100 times greater, preferably at least 1000 times greater, more preferably at least 1×10.sup.4 times greater, and still more preferably at least 1×10.sup.5 times greater than occurs in vivo, and exists substantially free from other macromolecules or biologically-active small molecules that occur together with the macromolecule or small molecule of interest in vivo.

Methods for preparing large libraries of compounds, including simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources.

The number of different compounds to test in the methods of the invention will depend on the application of the method. For example, one or a small number of test compounds can be advantageous in manual screening procedures, or when it is desired to compare efficacy among several predicted ligands, agonists or antagonists. However, it is generally understood that the larger the number of test compounds, the greater the likelihood of identifying a compound having the desired activity in a screening assay. Additionally, large numbers of compounds can be processed in high-throughput automated screening assays. Therefore, “test compounds” can be, for example, 2 or more, such as 5, 10, 15, 20, 50 or 100 or more different compounds, which can be assayed simultaneously or sequentially.

When referring to the amino acid residue 620 of lymphoid tyrosine phosphatase (LYP), what is referred to is the residue position on an LYP peptide, or variant thereof, that interacts with the tryptophan (W47) amino acid in the ligand binding cleft of the SH3 domain of wild type Csk. This LYP residue is an arginine in the wild-type LYP, and is not limited to amino acid position 620 of LYP, but rather can be in any of a variety of amino acid positions, for example in the case of a truncated LYP, a splice variant LYP, a fusion protein with LYP or other similarly configured variants. In this sense the term “. . . typically residue 620 of lymphoid tyrosine phosphatase . . . ” or the term “. . . amino acid residue 620 . . . ” or a similar term may be used herein.

When referring to the nucleic acid residue 1858 of the PTPN22 gene, what is referred to is the nucleic acid in codon 620 that codes for an arginine in amino acid position 620 of the full-length LYP peptide. Similar to the above, the nucleotide may be in other actual positions, as will be the case when coding for a fragment, fusion or variant of the LYP protein. However, this nucleic acid residue codes for an amino acid residue in the LYP peptide that is situated similarly with respect to its tertiary structure as the amino acid at position 620 of the wild type LYP, as used herein.

One embodiment of the present invention relates to the discovery of a single nucleotide polymorphism in the gene coding for lymphoid tyrosine phosphatase. This SNP causes a variation in amino acid sequence for the lymphoid tyrosine phosphatase protein, which subsequently leads to a variety of autoimmune disorders.

In one aspect of the invention there is provided a diagnostic screening method to determine the presence of in vivo characteristics leading to an autoimmune disorder in a mammal comprising the steps of: (a) isolating an in vivo component from a person to be screened for a characteristic leading to an autoimmune disorder; (b) performing a screening assay using said isolated in vivo component; (c) comparing a result derived from the screening assay with a known value for that characteristic; (d) correlating said in vivo component to a known characteristic leading to an autoimmune disorder, such that the presence of at least one characteristic indicates an individual's susceptibility to an autoimmune disorder, relating to said in vivo component and stemming from dysregulation of the immune response system.

In a further aspect of the current invention there is provided a method of screening for agents that modulate lymphoid tyrosine phosphatase mediated immune system regulation comprising the steps of: (a) providing a system further comprising: (i) a peptide similar to Csk that can specifically interact with a peptide similar to lymphoid tyrosine phosphatase; (ii) at least one peptide similar to lymphoid tyrosine phosphatase and at least having an amino acid that is situated substantially similarly with respect to its tertiary structure as the amino acid at position 620 in wild-type lymphoid tyrosine phosphatase protein is situated; and (iii) a reporter system to report the interaction of the peptide similar to Csk with the peptide similar to lymphoid tyrosine phosphatase; (b) introducing a test compound to the system; and (c) determining the effect that the test compound has on the system.

In a further aspect of the current invention there is provided a modulator of lymphoid tyrosine mediated T-cell regulation.

In a further aspect of the current invention there is provided a method for the treatment of an autoimmune disease comprising the steps of administering to a patient diagnosed with an autoimmune disease an agent that modulates the consequences of dysfunctional lymphoid tyrosine phosphatase protein on the lymphoid tyrosine phosphatase mediated regulation of the immune system in a quantity sufficient to supplement lymphoid tyrosine phosphatase mediated regulation of the immune system and, thereby treat autoimmune disease.

In a further aspect of the current invention there is provided a method for the treatment of an autoimmune disease comprising the steps of administering to a patient diagnosed with an autoimmune disease an exogenous nucleic acid molecule that modulates the consequences of dysfunctional lymphoid tyrosine phosphatase protein on the lymphoid tyrosine phosphatase mediated regulation of the immune system in a quantity sufficient to supplement lymphoid tyrosine phosphatase mediated regulation of the immune system and, thereby treat autoimmune disease.

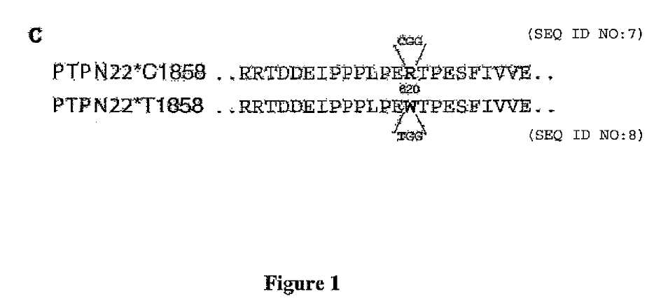

Applicants have found that the human PTPN22 gene contains a SNP at nucleotide 1858 in codon 620, which encodes an arginine (CGG Seq. ID No.: 1) in both alleles of the PTPN22 gene (“PTPN22*R1858”) for the wild-type protein in all published human and mouse Lymphoid Tyrosine Phosphatase (“LYP”) sequences, but encodes a tryptophan (TGG Seq. ID No.: 2) in at least one allele of the PTPN22 gene (“PTPN22*T1858”) leading to a mutant LYP protein (FIG. 1). The nucleic acid sequences and the amino acid sequences of wild type lymphoid tyrosine phosphatase and orthologues thereof is known and documented, as are the sequences of splice variants of these proteins (See e.g., United States Patent Application Number 2004/0006777, by Roifinan, Chaim M., which is incorporated herein by reference). The mutant PTP gene is Accession No.: AL137856, found at Genbank (http://www.ncbi.nlm.nih.gov/entrez/viewer.fcgi?db=nucleotide&val=14970654).

In one study, Applicants determined that approximately 16.2% of individuals from the North American population were found to harbor the PTPN22*T1858 allele (Table I). Only four of 395 healthy individuals were homozygous for the PTPN22*T1858 allele. The role of LYP is as a gatekeeper in T cell activation (“TCA”), acting to suppress TCA. Thus Applicants have discovered that the SNP resulting in the PTPN22*T1858 allele alters the functions of the phosphatase in TCA.

TABLE I
Frequency of PTPN22 genotypes in TID* two independent samples.

| Genotypes^{b}, percentage (n) | | |
|---|---|---|
| *C*C | *C*T | *T*T |

North American samples^{a}

| | | | |
|---|---|---|---|
| TID^{c} (n = 294) | 65.6 (n = 193) | 30.6 (n = 90) | 3.7 (n = 11) |
| Controls^{d} (n = 395) | 77.7 (n = 307) | 21.3 (n = 84) | 1.0 (n = 4) |

χ² = 14.84, df = 2, p < 0.001; OR, 1858T carrier vs. non-carrier = 1.83 (95% ci = 1.284-2.596)

Italian sample^{a}

| | | | |
|---|---|---|---|
| TID^{e} (n = 174) | 90.8 (n = 158) | 8.6 (n = 15) | 0.6 (n = 1) |
| Controls^{f} (n = 214) | 95.8 (n = 205) | 4.2 (n = 9) | 0 (n = 0) |

χ² = 3.93, df = 1, p < 0.05; OR, 1858T carrier vs. non-carrier = 2.31 (95% ci = 0.932-5.819)
*TID - Insulin-dependent type I diabetes mellitus.
BEGIN LEGEND FOR TABLE 1 (A-F below)
^{a}The study was approved by the Institutional Review Boards of the Institutions where the subjects were recruited. The nature of the study was explained to study participants and informed consent was obtained by each patient and control subject.
^{b}Genotypes were determined as described in FIGS. 1-4 herein.
^{c}This TID sample consisted of unrelated, non-Hispanic, Caucasian subjects diagnosed with type I diabetes at the Barbara Davis Center for Autoimmune Diabetes, Denver, CO
^{d}This control sample consisted of 189 unrelated, non-Hispanic, Caucasian, college students from California, 160 healthy unmarried, unrelated, non-Hispanic, Caucasian parents of the Minnesota Twin Study, and 46 healthy, unrelated, non-Hispanic, Caucasian controls from the Barbara Davis Center for Autoimmune Diabetes, Denver, CO. These samples showed no statistically significant differences between each other.
^{e}This TID sample consisted of unrelated subjects diagnosed with type I diabetes at the Department of Pediatrics of the University of Sassari, Italy.
^{f}This control sample consisted of healthy individuals from the same population as in E.

END LEGEND FOR TABLE 1

Insulin-dependent type I diabetes mellitus (TID) affects 0.5% of the human population and approximately 1.4 million people in the United States. The disease is believed to arise as the consequence of an autoimmune destruction of insulin-producing .beta.-cells by cytotoxic CD8 T cells with CD4 T cell help. Using the discovery that the PTPN22*T1858 allele leads to a disruption in LYP's modulatory role and subsequent dysregulation of TCA, there was designed and performed a genetic association study using a sample of 294 Caucasian patients with TID and 395 healthy subjects from the same ethnic background. Subjects were genotyped, and it was discovered that the PTPN22*T1858 allele was more frequent in TID patients than in healthy individuals: 30.6% of the TID patients were of the *C*T genotype compared to only 21.3% of the healthy controls. Furthermore, 3.7% of the TID patients were homozygous for the *T1858 allele, compared to only 1.0% of the healthy controls. (_{χ}=14.84 with 2 degrees of freedom, p<0.001; OR for the *T carrier versus non-carrier=1.83, 95% confidence interval=1.284-2.596). The cohorts were matched for age and race. These results lead to the discovery that the PTPN22*T1858 allele predisposes an individual for developing TID.

There was also genotyped a second, completely independent and ethnically separate cohort of 388 individuals from Sardinia, Italy. All patients and controls came from northern Sardinia, a region characterized by an ethnically homogenous population. In this study, more than twice as many of the TID patients as controls were heterozygous, 8.6% versus 4.2%, respectively, and the only homozygous *T*T individual found in the cohort had the disease. (_{χ}2=3.93 with 1 degrees of freedom, p<0.05; OR for the *T carrier versus non-carrier=2.31, 95% confidence interval=0.932-5.819). The discovery of the predisposition to TID conferred by the PTPN22*T1858 allele is present in these different backgrounds where the allele frequency is lower.

Because LYP is expressed in lymphocytes and T1D and other autoimmune diseases are caused by dysfunctional lymphocytes, the current discovery correlating the mutant genotype/phenotype with the altered lymphocyte function lead to the understanding that LYP plays a possible role in numerous systemic autoimmune diseases, in addition to T1D. For example, it is known that the PTPN22 gene resides at chromosomal region 1p13, which has been linked to systemic lupus erythematosus and rheumatoid arthritis. LYP has also been associated with juvenile arthritis, Graves disease and Addison disease. Thus, lymphoid tyrosine phosphatase is useful for the control of numerous systemic autoimmune disorders.

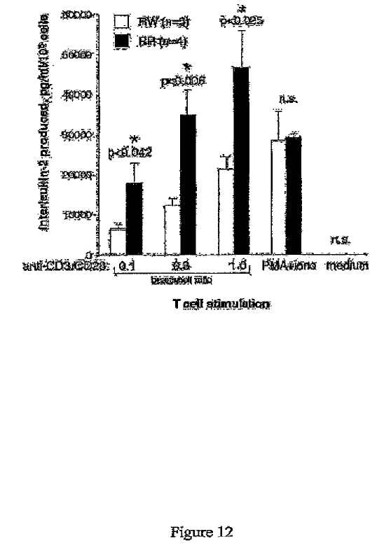

It has also been determined that the putative disease-predisposing variant of LYP, LYP*W620, impacts T cell behavior in a way that can be measured by functional or biochemical assays. First determined was the activation of primary T lymphocytes from genotyped T1D patients. For this purpose, 631 Sardinian T1D families were genotyped. In this sample (which was independent from the one reported above), the association of C1858T with T1D, evaluated by the extended transmission disequilibrium test, was significant: transmission of the W*620 allele was 69.4% (p=0.0033). T cells isolated from five unrelated carriers of the autoimmunity-predisposing LYP*W620 allele (RW genotype) secreted significantly less interleukin-2 than T cells from 4 unrelated patients of RR genotype (FIG. 12). This was seen at three different ratios of antibody-coated beads to cells in the T cell activation assays. As an important control, the response to phorbol ester plus ionomycin was similar between the two groups. Thus, there was a clear difference in the response to TCR stimulation between the carriers of LYP*W620 and non-carriers, but not in response to pharmacological activation that bypasses early TCR signaling events.

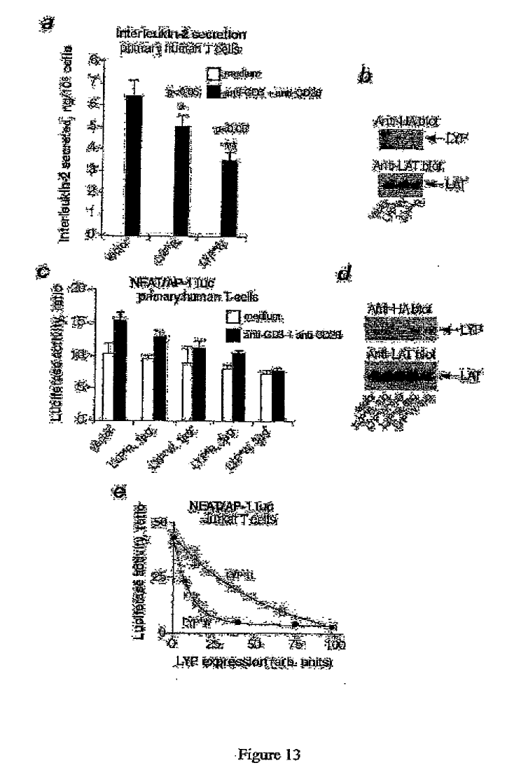

Next determined was the activation of primary T lymphocytes transfected with physiological amounts of LYP*W620 or LYP*R620. Transfection efficiency using the nucleofection technique (Amaxa Inc., Germany) was consistently 60-80% of cells, allowing us to analyze the cells directly without sorting. Stimulation of these cells with anti-CD3ε plus anti-CD28 mAbs led to production of interleukin-2, which was reduced by LYP (FIGS. 13a and 13b). At similar amounts of expressed protein, LYP*W620 consistently inhibited the interleukin-2 response to a higher extent. Thus, the disease predisposing LYP*W620 was a more efficient inhibitor of T cell activation.

Similar results were obtained with expression of the disease-predisposing LYP*W620 or the normal LYP*R620 in primary T cells (FIGS. 13c and 13d) or Jurkat T cells (FIG. 13e) together with a luciferase reporter gene driven by the nuclear factor of activated T cells (NFAT)/activator protein-1 (AP-1) transcription factor complex. Stimulation of these cells with anti-CD3ε plus anti-CD28 mAbs revealed that both LYP alleles inhibited the response in a dose-dependent manner. However, LYP*W620 inhibited the response at much lower levels of protein expression than LYP*R620 (FIG. 13c-e). At similar amounts of transfected DNA, LYP*W620 inhibited the luciferase reporter to a higher extent. Also normalized for expression levels, the dose-response for LYP*W620 was clearly shifted to the left compared to that for LYP*R620, suggesting again that LYP*W620 was a more efficient negative regulator of T cell signaling than LYP*R620.

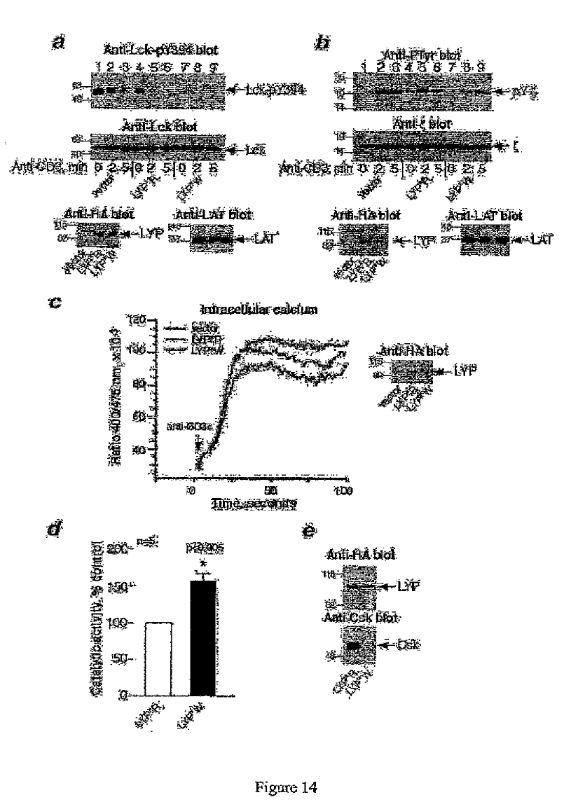

To address this more directly, the tyrosine phosphorylation of proteins involved in the earliest events of TCR signaling was determined. In cells expressing LYP*W620, the time-course of Lck phosphorylation at its autophosphorylation site, Y394, was clearly inhibited compared to T cells expressing LYP*R620 (FIG. 14a). Lck-mediated phosphorylation of the TCRζ chain was similarly reduced (FIG. 14b). TCR-induced calcium mobilization was also inhibited more by LYP*W620 than by LYP*R620 (FIG. 13c). Thus, LYP*W620 again was more efficient than LYP*R620 when compared at equal levels of expression.

Finally, the catalytic activities of LYP*W620 and LYP*R620 immunoprecipitated from cells was measured. A phosphopeptide modeled after the autophosphorylation site of Lck, a physiological substrate, was used as a substrate. (See Gjörloff-Wingren, A., Saxena, M., Williams, S., Hammi, D. & Mustelin, T. Characterization of TCR-induced receptor-proximal signaling events negatively regulated by the protein tyrosine phosphatase PEP. Eur. J. Immunol. 29, 3845-3854 (1999).) Immunoprecipitated LYP*R620 dephosphorylated 6.312±1.596 pmoles of substrate/min/106 transfected cells (n=5), while LYP*W620 displayed a 57.2% (±9.8%, n=5) higher specific activity when corrected for amount of protein (FIG. 14d), despite not binding Csk (FIG. 14e). These direct measurements provided the final proof that the *W620 allele encodes a more active phosphatase.

So, the LYP variant that causes autoimmune disease was determined to be a gain-of-function form of the enzyme for these above assays. Although a simplistic model of autoimmunity would predict that T cells with defects that augment TCR signaling would be likely to cause disease, experiments have shown that peripheral T cells from T1D patients rather are hyporesponsive to in vitro stimulation with anti-CD3 antibodies. Thymocytes from non-obese diabetes mice are also hyporesponsive to TCR-mediated activation and proliferation, and indirect evidence suggests that thymocyte hyporesponsiveness due to anomalies in early TCR signaling plays a causative role in autoimmune disease.

The increased efficacy of LYP*W620 to inhibit TCR signaling may lead to weaker signaling and therefore a failure to delete autoreactive T cells during thymic selection and/or insufficient activity of regulatory T cells. This is one explanation of why LYP*W620 increases the susceptibility to a number of diseases and why even one mutated allele confers predisposition to T1D and other autoimmune disorders.

LYP*W620 is a gain-of-function mutant having nearly twice the enzymatic activity of the wild type. In this aspect, therefore, there is provided a screening method for identifying small molecules that inhibit or reduce the mutant LYP enzymatic activity. Identified inhibitory modulators are useful to prevent the emergence, or reappearance, of autoreactive T cells.

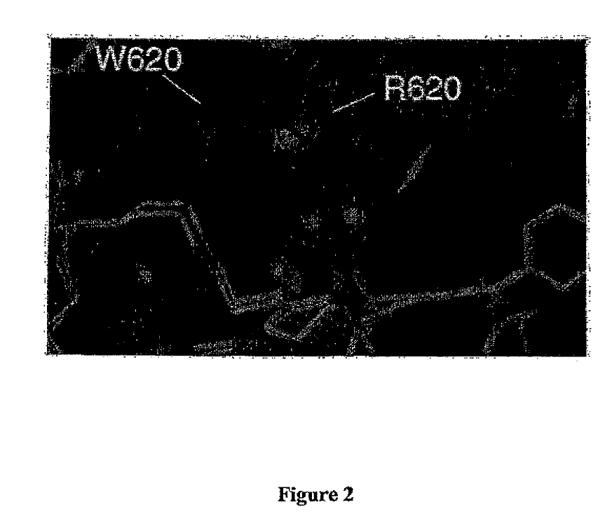

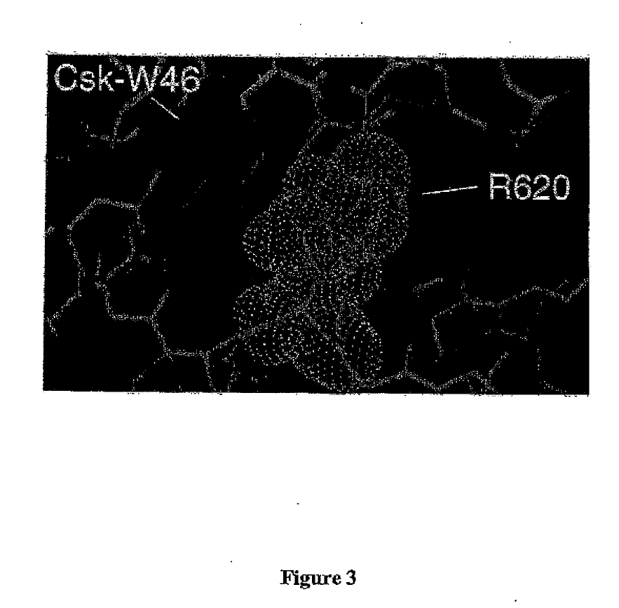

To determine the molecular basis of the correlation between the PTPN22*T1858 allele and autoimmune disorders, inventors examined the location and role of the amino acid encoded by codon 620. Codon 620 codes for a residue residing in the P1 proline-rich motif of LYP, which is involved in binding the SH3 domain of Csk during suppression of TCA (FIG. 1). The three-dimensional structure of this complex shows that the side chain of R620 fits into a somewhat acidic depression within the ligand binding cleft of Csk's SH3 domain (FIG. 2), where it interacts with a tryptophan residue (W47) of Csk (FIG. 3). Replacing R620 with a tryptophan residue disrupts this interaction by replacing the smaller, polar (basic) arginine residue with a bulkier, non-polar tryptophan residue. The bulkier, non-polar side chain of tryptophan will not fit into the pocket. Similarly, a polymorphism very near residue 620, particularly one affecting the proline residues, can cause a structural change in LYP such that the R620 residue is unable to properly contact the ligand binding cleft of the Csk SH3 domain. Still further, an intronic polymorphism in the nucleic acid may affect the transcription and/or translation of the LYP protein such that LYP is unable to interact with Csk. Such polymorphisms will also prevent LYP and Csk interaction leading to numerous autoimmune disorders, including, but not limited to type-1-diabetes, systemic lupus, erythematosus, juvenile arthritis, rheumatoid arthritis, Graves disease and Addison disease.

Without being bound by any theory, the current model is that both LYP and Csk affect T-Cell Activation by acting on Src family kinases. In particular LYP would dephosphorylate the positive regulatory tyrosine in the active site of the Src-family kinase, while Csk would phosphorylate the negative regulatory tyrosine at the C-term of the Src-family kinase. Both LYP and Csk taken alone are negative regulators of T-Cell activation, but they act synergistically in the complex. Thus, disruption of the complex results in an overall increase in T-Cell Activation.

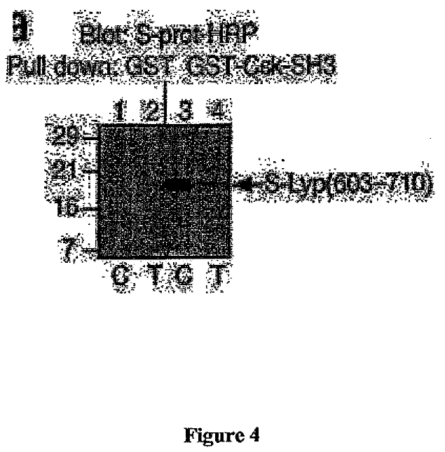

Applicants prepared a series of constructs having a fragment of LYP comprising amino acids 603-710 and expressed as an S-tagged protein using E. coli. The s-tagged constructs were prepared using pET-30, (Novagen, San Diego, Calif. 92121), however, other vector systems can be used. Construct 1, had an arginine at position 620; while construct 2 had a tryptophan at position 620. The constructs were equally expressed using E. coli BL21. Either a GST-Tagged SH3 domain of Csk or a GST tag alone was co-expressed with the LYP fragment constructs. A glutathione-agarose bead was added to each of the BL21 lysates. Precipitated proteins were separated by gel-electrophoresis and transferred to a nitrocellulose filter. The filters were incubated with S-protein HRP and ECL reagent to allow for chemiluminescent detection of Csk bound LYP. (ECL Detection Reagents, GE Health Care, formerly Amersham Biosciences, Piscataway, N.J. 08855.) The construct with arginine at position 620 precipitated in a pull down assay using the GST-SH3 domain of Csk (FIG. 4), whereas the constructs having tryptophan at position 620 did not precipitate. None of the constructs precipitated when co-expressed with GST alone. In this example, bound material was detected using S-protein-horseradish peroxidase.

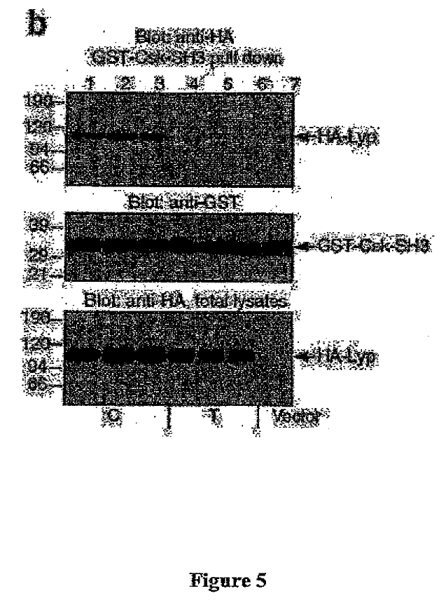

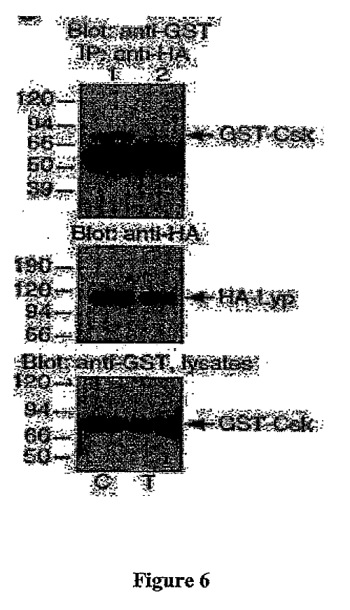

Similarly, full-length LYP with R620 (*C1858 allele) expressed in COS cells readily precipitates using the Csk SH3 domain, while LYP with W620 (*T1858 allele) does not (FIG. 5). In this example, Applicants expressed in COS cells full length Hemagglutinin (HA) tagged LYP having either R620 or W620 using pEF-HA. (See, Huynh, H et al., J. Immunol., 171: 6662 (2003).) The cells were lysed and the lysates were incubated in the presence of GST-SH3/Csk, and were subjected to common pull down assay techniques. Bound material was detected using anti hemagglutinin antibody in a two stage antibody detection procedure having an anti-HA primary antibody and an HRP labeled anti-mouse secondary antibody, and using ECL reagent. The top panel of FIG. 6 shows that only LYP R620 bound the GST-SH3-Csk: LYP W620 did not. The center panel shows that equal amounts of GST-SH3/Csk were used in each pull-down, while the last panel shows that LYP was equally expressed in both the cells having wild type LYP and the cells having mutant LYP.

Full-length LYP with R620 and Csk also co-immunoprecipitate (FIG. 6). Applicants expressed Csk-GST and either LYP with R620 or LYP with W620 in COS cells. Cells were lysed using well known techniques and the lysates were immunoprecipitated with anti-hemagglutinin antibody. Three separate western blots were performed on the immunoprecipitated lysates, and the results are presented in FIG. 6. Using anti-GST-Csk antibody shows in the top panel that only the LYP R620 binds the SH3 domain of Csk. Using anti-hemagglutinin antibody shows that both the LYP R620 and the LYP W620 were expressed. A blot of total cell lysates using anti-GST-Csk antibody shows that the GST-Csk fragment was expressed in both cell lines.

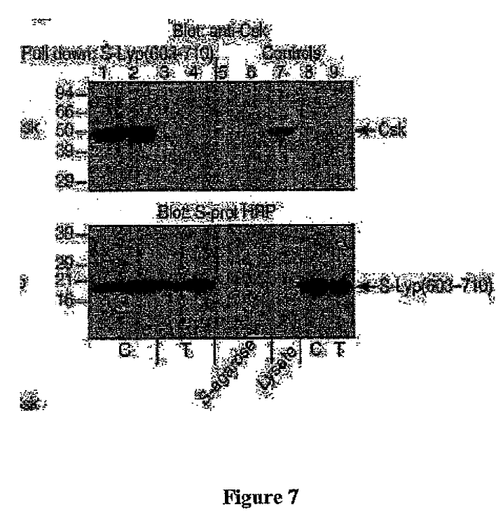

Endogenous Csk readily precipitates from T cells using S-tagged LYP peptide 603-710 with arginine, but not tryptophan, at position 620 (FIG. 7). Jurkat T cells, which endogenously express Csk, were lysed and the protein lysate was subjected to a pull down assay using S-tagged LYP either having R620 or having W620. A blot was performed and detection was carried out using an anti-Csk primary antibody in a two-stage detection system. Only the S-tagged LYP R620 bound with the endogenous Csk: the S-tagged LYP W620 did not.

Together, these results demonstrate that only LYP R620 (*C1858 allele) forms a complex with the Csk kinase, while LYP W620 (*T1858 allele) does not.

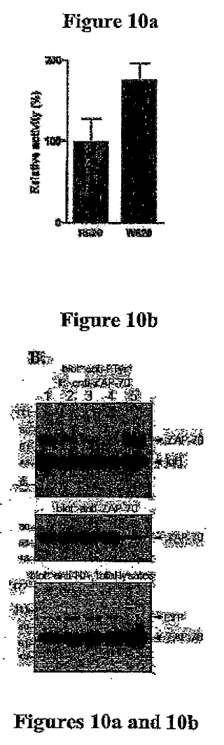

It has been further discovered that the phosphatase activity of LYP is increased in the mutant LYP W620 over the LYP R620 (FIG. 10a). Without being bound by any theory, the mutant LYP W620 does not associate with Csk (as discussed above), and thus the increased phosphatase activity of LYP W620 is taking place elsewhere. Applicants expressed Csk and either LYP R620 or LYP W620 in Jurkat cells. Both the LYP W620 and the LYP R620 were expressed with a hemagglutinin tag allowing for the amount of phosphatase in each reaction to be normalized. Expressed proteins from the transfected cells are immunoprecipitated and the enzymatic activity of each immunoprecipitate measured using p-Nitrophenyl Phosphate (pNPP) as a substrate. Detection of the relative amounts of phosphatase activity is determined at 405/620 nm (kinetic assay). FIG. 10a shows that the phosphatase activity of LYP W620 is significantly greater than the phosphatase activity of LYP R620.

LYP W620 is a more efficient inhibitor of Zap-70 auto-phosphorylation than is LYP R620. Zap-70 is a Syk-family protein tyrosine kinase expressed in T and Nk cells. Zap-70 plays a critical role in mediating T-Cell Activation in response to T-Cell receptor engagement. Following T-Cell receptor engagement Zap-70 is rapidly phosphorylated on several tyrosine residues, presumably by two mechanisms: an autophosphorylation and a transphosphorylation by the Src family tyrosine kinase Lck. Tyrosine phosphorylation of Zap-70 correlates with its increased kinase activity and couples to downstream signaling events. Phosphorylation of Tyr319 is required for the assembly of a Zap-70-containing signaling complex that leads to the activation of the PLC-g1-dependent and Ras-dependent signaling cascades in antigen-stimulated T cells.

Inventors co-expressed Zap-70 with Csk and with either 0.5, 1.0, or 1.5 .micro.g DNA of hemagglutinin tagged LYP R620; 2.0 .micro.g DNA of hemagglutinin tagged LYP W620 or pEF-HA vector alone in COS cells. See von Willebrand, M., Eur. J. Biochem., 1996, 235:828-835. The cells were lysed and the total lysates were separated and detected using well known westernblot techniques. In the upper and middle panels of FIG. 10b the immunoprecipitates are blotted with anti-Zap70 and anti-pTyr. In the lower pane, the blot is anti-HA on equal amounts of total cellular protein. FIG. 10b shows that LYP W620 is a more efficient inhibitor of Zap-70 autophosphorylation than is LYP R620.

These current findings suggest that a SNP in a lymphoid tyrosine phosphatase is a part of the complex genetic background of common autoimmune diseases, such as T1D. The SNP directly disrupts the formation of a LYP-Csk complex, the physiological relevance of which is well documented. Thus, individuals heterozygous for the PTPN22*T1858 allele have a reduced amount of LYP-Csk complexes. In addition, Applicants have shown that the mutant LYP W620 has greater phosphatase activity than does the wild type LYP R620. Thus, in addition to not forming a complex with Csk, the LYP W620 phosphatase has increased activity on other substrates. The result of this mutation in the lymphoid tyrosine phosphatase is a dysregulation of the immune system leading to an increase in autoimmune disorders.

Another aspect of the present invention is a substantially purified mutant lymphoid tyrosine phosphatase polypeptide having increased phosphatase activity. The mutant tyrosine phosphatase can be purified by standard protein purification techniques, including, but not limited to, ammonium sulfate precipitation, ion exchange chromatography, size exclusion chromatography, immunoprecipitation, affinity chromatography, chromatofocusing, electrofocusing, and other protein purification techniques well known in the art.

Because the increased phosphatase activity of the mutant lymphoid tyrosine phosphatase enzyme is associated with an abnormal phenotype that has susceptibility to autoimmune diseases, yet another aspect of the present invention is a screening method for compounds having the activity of inhibiting the increased phosphatase activity of the mutant lymphoid tyrosine phosphatase. Such compounds may have therapeutic usefulness in treating autoimmune diseases. In general, this method comprises the steps of:

(1) contacting a substantially purified mutant lymphoid tyrosine phosphatase enzyme having increased phosphatase activity with a compound that may inhibit the phosphatase activity of the substantially purified mutant lymphoid tyrosine phosphatase; and

(2) comparing the phosphatase activity of the substantially purified mutant lymphoid tyrosine phosphatase in the presence and in the absence of the compound, in order to determine whether or not the compound inhibits the phosphatase activity of the substantially purified mutant lymphoid tyrosine phosphatase activity.

Generally, the nomenclature used herein and the laboratory procedures in cell culture, molecular genetics, and nucleic acid chemistry and hybridization described are those well known and commonly employed in the art. Standard techniques are used for recombinant nucleic acid methods, polynucleotide synthesis, cell culture, and transgene incorporation (e.g., electroporation, microinjection, lipofection). Generally enzymatic reactions, oligonucleotide synthesis, and purification steps are performed according to the manufacturer's specifications. The techniques and procedures are also generally performed according to conventional methods in the art and various general references which are provided throughout this document, as well as: Maniatis et al., Molecular Cloning: A Laboratory Manual (1989), 2nd Ed., Cold Spring Harbor, N.Y.; and Berger and Kimmel, Methods in Enzymology, Volume 152, Guide to Molecular Cloning Techniques (1987), Academic Press, Inc., San Diego, Calif., which are incorporated herein by reference. Expression of recombinant proteins, protein binding, transfections, immunoprecipitation, and immunoblots were performed as described in detail in Saxena, M., Williams, S., Tasken, K. & Mustelin, T., Crosstalk between cAMP-dependent kinase and MAP kinase through hematopoietic protein tyrosine phosphatase (HePTP), Nature Cell Biology, 1, 305-311 (1999); and in Alonso, A. et al. Tyrosine phosphorylation of VHR by ZAP-70, Nature Immunol., 4, 44-48 (2002). Oligonucleotides can be synthesized on an Applied Bio Systems oligonucleotide synthesizer according to specifications provided by the manufacturer. The procedures are believed to be well known in the art and are provided for the convenience of the reader. All the information contained therein is incorporated herein by reference.

Methods for preparing large libraries of compounds, including simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources.

The number of different candidate compounds to test in the methods of the invention will depend on the application of the method. For example, one or a small number of candidate compounds can be advantageous in manual screening procedures, or when it is desired to compare efficacy among several predicted ligands, agonists or antagonists. However, it is generally understood that the larger the number of candidate compounds, the greater the likelihood of identifying a compound having the desired activity in a screening assay. Additionally, large numbers of compounds can be processed in high-throughput automated screening assays. Therefore, “one or more candidate compounds” can be, for example, 2 or more, such as 5, 10, 15, 20, 50 or 100 or more different compounds, which can be assayed simultaneously or sequentially.

### EXAMPLES

The following non-limiting examples are useful in describing the current invention, but in no way limit the current invention. Those of ordinary skill in the art will readily adopt the underlying principles of applicant's discovery to design a variety of assays without departing from the spirit of the current invention.

High Throughput Screening techniques are well known in the art and applicable to screening methods using Applicants' discovery. The number of different test compounds or in vivo characteristics used to test in the methods of the invention will depend on the application of the method. For example, one or a small number of test compounds can be advantageous in manual screening procedures, or when it is desired to compare efficacy among several predicted ligands, agonists or antagonists. However, it is generally understood that the larger the number of test compounds, the greater the likelihood of identifying a compound having the desired activity in a screening assay. Additionally, large numbers of compounds can be processed in high-throughput automated screening assays. Therefore, “test compounds” can be, for example, 2 or more, such as 5, 10, 15, 20, 50 or 100 or more different compounds, which can be assayed simultaneously or sequentially

Expression of LYP or fragments of LYP (such as the LYP catalytic domain, residues 603 to 710 or other fragments useful as described herein) can be via a baculovirus system. Baculovirally expressed LYP is useful in many of the following described examples and in the discussions of the current disclosure or for other uses by those of ordinary skill in the art. Those of ordinary skill in the art are familiar with the baculovirus expression system techniques and will readily utilize said expression system with the materials and methods of the current invention. These uses are well within the spirit of this current invention. (See, e.g., Invitrogen, Corp, Carlsbad, Calif., catalog nos.: 12562- and 10359-016; see also, BD Bioscience, La Jolla, Calif., catalog nos.: 554802, 554803, 554738 and 554740, as examples). Moreover, LYP or fragments of LYP can be expressed in bacteria cells, yeast cells or other expression hosts well know in the art. (See e.g., Brown, T. A., Gene Cloning and DNA Analysis, Blackwell 4 Ed., (2002); or Ausubel, F. M., Current Protocols in Molecular Biology, John Wiley and Sons, (2001).)

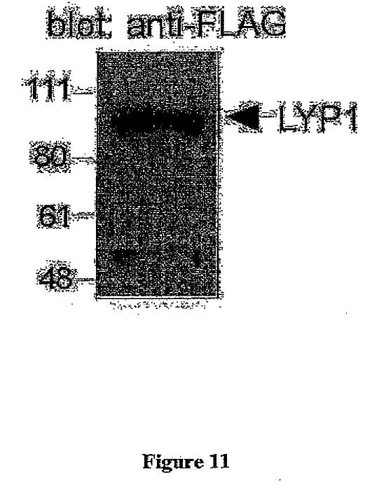

The following is a non-limiting example of cloned, expressed and isolated Flag-tagged LYP using a baculovirus system. In general, flag-tagged LYP R620 was expressed using a commercial baculovirus expression system (e.g., catalogue number 10359-016 from Invitrogen Corp., Carlsbad Calif.) and following manufacturer's instructions. The cell line sf9 was infected using a serial dilution of a recombinant baculovirus titer and varying incubation times. Expressed LYP R620 was isolated using Flag antibody conjugated beads and eluted by competitive dissociation using 0.2 mg/ml 3×FLAG peptide (e.g., catalogue numbers F3165 and F4799, respectively, Sigma, St. Louis, Mo.). The isolated LYP R629 was electrophoresed and detected using a two-stage detection system with an anti-flag first stage and a second stage having a detectable label was used following electrophoresis. The results are shown in FIG. 11. Thus, the baculovirus expression system is useful for techniques of the current invention.

### Example 1

Diagnostic Screening Assay

In a first example, the discovery is used to screen individuals to determine whether said individual carries the wild-type allele PTPN22*C1858 or the mutant allele PTPN22*T1858. Thus, in this example, the in vivo characteristic to be identified is an individual's genotype, particularly the PTPN22 gene, and more particularly nucleotide 1858.

In this example, an individual's white blood cells (“WBC”) are isolated and genomic DNA is extracted. A fragment of the PTPN22 gene is amplified using Polymerase Chain Reaction techniques (“PCR”). Briefly, 100 ng of total genomic DNA, 2.5 mM MgCl.sub.2 1× buffer and 5 U of AmpliTaq (Applied Biosystems, Foster City, Calif.) was combined with a sense primer 5′-TCA CCA GCT TCC TCA ACC ACA-3′ (SEQ. ID No.: 3) and antisense primer 5′ -GAT AAT GTT GCT TCA ACG GAA TTT A-3′ (SEQ. ID No.: 4) in a 25 microliter total reaction volume. The reaction was subjected to thirty cycles of the following parameters: 30″ at 95.deg.C.; 30″ at 60.deg.C.; and 30″ at 72.deg.C.

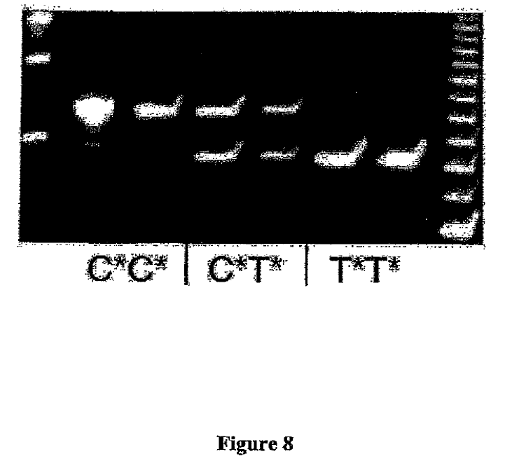

A restriction digestion of the PCR product identifies whether the individual harbors the wild-type allele or the mutant allele. The C to T transition at codon 620 creates in an Xcm I restriction site in the mutant allele, therefore the PCR products were incubated in a reaction mixture containg Xcm I (New England Bio Labs, Beverly, Mass., Cat. No.: R0533S) according to manufacturer's protocol for digestion. Each reaction mix is then resolved using a 3% agarose gel, and the electrophoresed product stained with ethidium bromide and viewed under ultra violate light. The range of results is illustrated in FIG. 8, wherein: columns 1 and 2 are typical results for a homozygous wild type individual; columns 3 and 4 are typical results for an individual heterozygous for the mutation; and columns 5 and 6 are typical results for an individual homozygous for the mutation. Based on the results from this example diagnostic assay, it can be determined whether an individual harbors one or more copies of the mutation, thereby being susceptible to autoimmune diseases.

### Example 2

Diagnostic Screening Assay

In a second diagnostic screening assay, individuals are genotyped to determine whether an individual carries the wild-type or mutant allele using well know sequencing techniques. In this example, the in vivo characteristic to be identified is an individual's genotype, particularly the PTPN22 gene, and more particularly nucleotide 1858.

In one example of this screening assay, the WBCs of an individual are isolated and genomic DNA extracted and PCR amplified as described in Example 1, above. The purified PCR product was sequenced using an automated sequencer, such as the ABI Prism 3100; however, any other sequencing procedure will reach the same result.

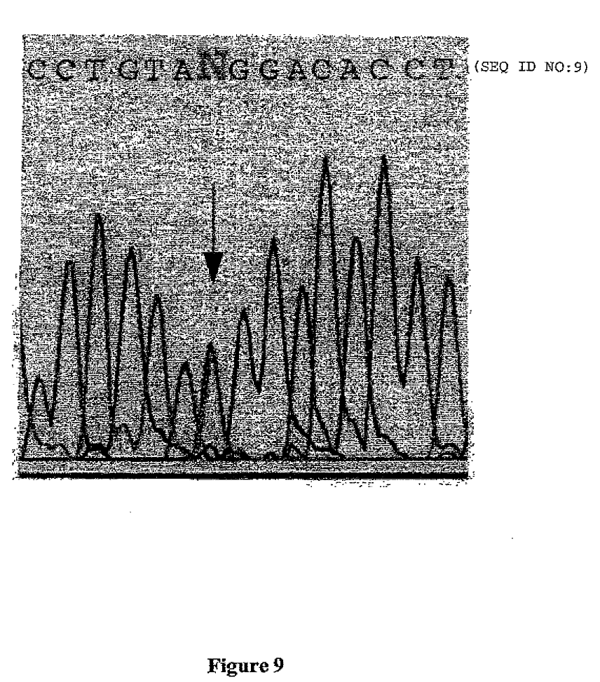

Individuals homozygous for the wild-type allele will have two copies of the sequence CGG at nucleotide positions 1858-1860, while individuals homozygous for the mutant allele will have two copies of the sequence TGG at nucleotide positions 1858-1860. Individuals having a heterozygous genotype will have a copy of each sequence (CGG and TGG) as listed above and the sequencing reaction will generate results similar to that shown in FIG. 9, wherein the arrow indicates both a C and a T detected at position 1858.

### Example 3

Diagnostic Screening Assay

A further variation of the diagnostic screening assay includes resolving the sequence of an individual's endogenous LYP protein. Thus, in this example, the in vivo characteristic to be identified is an individual's phenotype, particularly the amino acid sequence of the lymphoid tyrosine phosphatase protein, more particularly the amino acid sequence of the P1 domain of the lymphoid tyrosine phosphatase protein, and most particularly amino acid residue 620 of the lymphoid tyrosine phosphatase protein.

The LYP protein sequence can be resolved using common protein sequencing techniques (for example, using the CLC Capillary 494 Sequencer from Applied Biosystems), or using well known binding assays, such as pull-down assays or western blots to name a few.

For example, the source of LYP is the WBCs, which can be isolated from whole blood using well known techniques. Isolated WBCs are then lysed to release the cellular protein, and LYP is separated from total cellular protein using an antibody capture assay having antibodies specific for LYP.

Aliquots of LYP isolated from an individual is added to a diagnostic assay plate having wells either containing antibody specific for LYP R620 or specific for LYP W620. Methods for immobilizing an antibody to a plate are well known in the art. One example includes employing a GST tag fused to one end of an antibody. GST fused antibodies are easily immobilized on glutathione-bound plastic surfaces or beads through the interaction between the GST portion and its substrate, GSH. The assay plate will have antibodies specific for LYP R620 in one well and antibodies specific for W620 in other wells.

The aliquots of LYP protein isolated from an individual are incubated for a sufficient time to allow for reaction with the antibodies. The wells are washed to remove unbound protein and then the presence or absence of bound LYP is determined using a two stage antibody detection method having a first antibody raised against LYP and having a second antibody raised against the first and having a detectable label.

Individuals with wild-type LYP protein will only have LYP detected in the wells having immobilized antibody directed towards the LYP R620. Conversely, individuals having the mutant LYP protein will only have LYP detected in the wells having immobilized antibody directed towards the LYP W620. Heterozygous individuals will have LYP detected in wells having both LYP R620 and LYP W620.

### Example 4

Modulator Screening Assay

In one screening assay, modulators of the LYP-SH3/Csk mediated regulation of T-Cell Activation (TCA) are discovered. The method of this screen includes exposing cells to a variety of test compounds and assaying for LYP binding at the SH3 domain of Csk.

WBCs are isolated from individuals homozygous for LYP R620 and from individuals homozygous for LYP W620, as well as individuals heterozygous for LYP R620/W620. Each cell line is plated in separate wells of a tissue culture plate and then each well is incubated in the presence or absence of a test compound (LYP R620 cells with test compound; LYP R620 without test compound; LYP W620 cells with test compound; LYP W620 without test compound; LYP R620/W620 with test compound and LYP R620/W620 without test compound). Control wells for each cell line are those indicated above as being “without test compound” and will receive reaction mixture alone without modulator to establish a base line binding.

Following incubation, the cells are lysed and the cellular protein isolated, separated and detected using common western blot techniques. Two-stage antibody detection is preferably used having a first antibody raised against Csk and a second stage antibody raised against the first and labeled with a detection enzyme. Csk/LYP will blot at a different position on the nitrocellulose than will Csk alone, due to variations in migration during electrophoresis. Migration position will disclose whether or not LYP/Csk interaction occurred in the presence of test compound. Changes in LYP binding to SH3/Csk in the presence of test compounds are detectable relative to the control wells.

Modulators that increase the binding of LYP R620 with the SH3 domain of Csk will show an increase over the control wells and are useful for developing treatments to increase the binding and actions of LYP R620 in heterozygous individuals. Modulators that decrease the binding of LYP R620 are useful for the developing treatments to reduce overactive LYP R620 binding with SH3/Csk. Modulators that force the interaction of LYP W620 with SH3/Csk are useful for developing treatments for the regulation of T-Cell Activation in individuals having both the heterozygous and the homozygous mutant genotypes.

### Example 5

Modulator Screening Assay

In another screening assay, modulators of the LYP-SH3/Csk mediated regulation of T-Cell Activation (TCA) are discovered. The method of this screen includes transfecting a cell line with a fragment of Csk having at least the SH3 domain and having a GST tag, then exposing the transfected cells to a variety of test compounds and assaying for test compound mediated LYP binding at the SH3 domain of Csk.

Cell lines that do not express endogenous Csk and that have either a homozygous LYP R620 phenotype, a homozygous LYP W620 phenotype, or a heterozygous R620/W620 phenotype are plated in separate wells. Each transfected cell line is plated in separate wells of a tissue culture plate and then each well is incubated in the presence or absence of a test compound (LYP R620/R620 cells with test compound; LYP R620/R620 without test compound; LYP W620/W620 cells with test compound; LYP W620/W620 without test compound; LYP R620/W620 with test compound and LYP R620/W620 without test compound). Control wells for each cell line are those indicated above as being “without test compound” and will receive reaction mixture alone without modulator to establish a base line binding.

Following incubation, the cells are lysed and the cellular protein is separated using well known western blot techniques. A two stage antibody detection step includes a first antibody is raised against the GST tag and a second antibody raised against the first and having a detection label. Changes in LYP binding to SH3/Csk in the presence of test compounds are detectable relative to the control wells.

Modulators that increase the binding of LYP R620 with the SH3 domain of Csk will show an increase over the control wells and are useful for developing treatments to increase the binding and actions of LYP R620 in heterozygous individuals. Modulators that decrease the binding of LYP R620 are useful for the developing treatments to reduce overactive LYP R620 binding with SH3/Csk. Modulators that force the interaction of LYP W620 with SH3/Csk are useful for developing treatments for the regulation of T-Cell Activation in individuals having both the heterozygous and the homozygous mutant genotypes.

### Example 6

Modulator Screening Assay

In a further screening assay, modulators of the LYP/Csk mediated regulation of Src family proteins are determined. The method of this screen includes measuring the phosphatase activity of LYP on a substrate and/or the kinase activity of Csk on a substrate. Preferably, in this example screening assay the substrate is a Src family member, such as Lck; however, a variety of substrates including synthetic substrates can be used.

One source of the LYP, Csk and/or Src family member proteins is from white blood cells (WBC). WBCs can be isolated from individuals homozygous for LYP R620 and from individuals homozygous for LYP W620. The cells can be lysed and the proteins separated and isolated using any of a number of well known techniques. The proteins are then placed into wells of a multi-well plate such that the wells either comprise LYP R620, Csk and a substrate; or LYP W620, Csk and a substrate. Test compounds are added to the wells such that any single test compound is added to a well having LYP R620 and to a well having LYP W620. Baseline activity is determined by providing wells either comprising LYP R620, Csk and a substrate; or LYP W620, Csk and a substrate; both without test compounds. Also included on the multiwell plate is a series of control wells. Depending on the detection system to be used, the composition of the control wells will vary. In a preferred embodiment of this example, the detection method is a phosphatase assay and the control wells are a serial dilution of phosphate standard as well as a blank. Detection of phosphatase activity in the test wells is compared to the standard curve and is quantified using linear regression. Other detection methods exist, including, but not limited to western blots using first stage antibody raised against the phosphorylated species of substrate (e.g., Phospho-Lck Tyr494)

As stated above, LYP and Csk work in conjunction to regulate T-Cell Activation. The LYP phosphatase activity is measured as an indication of LYP action in the T-Cell signaling pathway, while Csk kinase activity can be measured as an indication of the Csk action in the T-Cell signaling pathway. For wells having no test compounds, the baseline phosphatase activity of LYP R620 and LYP W620 is determined. In addition, the baseline kinase activity of Csk can be determined in these wells. Test compounds that modulate the phosphatase activity of LYP R620 or LYP W620 are useful as phosphatase inhibitors and stimulators acting in the T-Cell Activation signaling pathway. The degree of modulation can be determined through linear regression across the standard curve. The modulators of this assay are useful for developing compounds that stimulate of inhibit phosphatase activity in the T-Cell Activation pathway.

### Example 7

Modulator Screening Assay

In a further screening assay, a population of human white blood cells is screened and cells having a homozygous LYP W620 phenotype are selected. The cells are transformed with an inducible vector having LYP R620, and are assayed for LYP mediated T-Cell activation.

Jurkat T cells made homozygous for LYP W620 are transformed with one of a variety of vectors having an inducible LYP R620 insert. The vectors will vary to comprise a variety of elements, including, but not limited to the vector backbone, promoter elements and other regulatory elements, as well as the inserted nucleic acid of choice. The transformed cells are plated in a multi-well plate. Included in the multi-well plate along with the transformed cells are cells homozygous for LYP R620, which are used as control wells to determine T-Cell activation levels for endogenous LYP R620 in the presence of a modulator.

The plated cells are incubated in a reaction mixture having either a known modulator of LYP induced T-Cell Activation or having reaction mixture alone. Following incubation, the cells are lysed and the cellular protein is isolated, separated and detected using well known western blot techniques. The detection step can be a variety of well known methods, including, but not limited to two stage antibody detection. The first stage antibody can be raised against the LYP-SH3/Csk interaction site, such that this first antibody only binds to LYP bound SH3/Csk domains. The second stage antibody is raised against the first and has a detectable label.

Wells having transformed cells showing an increase in the binding of LYP to SH3/Csk in comparison to wells having no test compound are useful for developing gene therapy treatments to down regulate T-Cell activation by introducing exogenous LYP R620. The efficiency of exogenous LYP R620 binding with SH3/Csk can be determined by comparison of the transformed cells to the cells having endogenous LYP R620. For example, transformed cells that are determined to have been transformed with a vector/LYP R620 construct that is capable of regulating T-Cell activation through LYP in the presence of a known modulator of T-Cell activation can be compared to cells having endogenous LYP R620. The efficiency of the exogenous LYP-SH3/Csk binding is determined by comparison to the levels of LYP-SH3/Csk binding in the endogenous cells.

### Example 8

Anti-Sense Nucleic Acid Therapy

In a further assay, WBCs having either the homozygous genotype expressing LYP R620 or the heterozygous genotype expressing LYP R620/W620 are plated and transformed. Each of the plated cell lines are transformed with either a pEF-HA vector expressing an anti-sense nucleic acid, preferably an anti-sense RNA specific for the RNA sequence expressing and flanking the gene segment of PNPN22*T1848, with the pEF-HA vector alone; or with no vector (reaction mix only). The anti-sense RNA molecules screened in this system each cover the SNP but have a variety of flanking sequences. The variety of flanking sequences is useful for determining an anti-sense RNA molecule that is specific for LYP W620 and not LYP R620. Antisense RNA that is specific for the LYP W620 transcript will free up the cellular translation machinery, thereby allowing for abundant production of LYP R620 in the heterozygous cell type. Furthermore, the more sensitive these antisense RNA molecules are for the LYP W620, the more thoroughly translation of the mutant copy is blocked. Transformed cells are then incubated in the presence or absence of a compound known to induce the binding of LYP with SH3/Csk.

Following incubation of the cells in the presence of an inducer compound, the cells are lysed and the cellular protein is isolated, separated and detected using well known western blot techniques. Detection takes place using a two stage anti-HA antibody (Covance, Princeton N.J.) technique with the first anti-HA antibody being raised against phosphor Tyr494 Lck, and the second anti-HA antibody is raised against the first with a detectable element attached. Analysis of the results includes comparing wells having the heterozygous genotype to wells having the homozygous genotype. Heterozygous wells having Lck phosphorylation/dephosphorylation levels similar to that in the homozygous wells have efficient anti-sense RNA binding. In addition, the wells are compared to the wells having no vector, assuring that the vector itself is not interfering with LYP mediated T-Cell regulation. Expressed anti-sense RNA that efficiently blocks translation of LYP W620 without interfering with translation of LYP R620 are useful for developing anti-sense RNA treatments.

In a variation of this example, homozygous LYP W620 cells are plated and transformed as above; however, also included is a vector expressing LYP R620. These cells are assayed as stated above. The results from these cellular assays are useful for developing treatments wherein both wild-type LYP is introduced and wherein the mutant LYP is blocked from translation, thereby allowing for the translation of the wild-type LYP.

### Example 9

In a further screening assay, the phosphatase activity of lymphoid tyrosine phosphatase on a substrate is determined. In this example, the substrate is a synthetic compound, preferably p-NitroPhenyl Phosphate (Rainbow Scientific, Inc, Windsor, Conn. 06095, Cat. No. 4400A).

Applicants expressed Csk and either LYP R620 or LYP W620 in Jurkat cells. Both the LYP W620 and the LYP R620 were expressed with a hemaglutinin tag allowing for the amount of phosphatase in each reaction to be normalized, (e.g., vector pEF-HA as described above). Expressed proteins from the transfected cells are immunoprecipitated using anti-HA antibody (Covance, Princeton, N.J.), and the immunoprecipitants are added to individual wells of a multi-well plate in the presence or absence of a test compound. The wells having no test compound are useful for determining basal level phosphatase activity. In addition, a well having no LYP but having substrate is useful as a negative control. The LYP phosphatase activity of each immunoprecipitate in the presence or absence of a test compound is measured using p-NitroPhenyl Phosphate (pNPP) as a substrate. Detection of the relative amounts of phosphatase activity is determined at 405/625 mn (kinetic assay), and the results are compared. The results of this kinetic assay are useful for discovering inhibitors and stimulators of the LYP phosphatase activity, and are also useful for discovering test compounds that reduce the phosphatase activity of the mutant LYP W620 to more near that of the wild-type LYP R620, and the converse as well.

### Example 10

In a further screening assay, the phosphatase activity of recombinant lymphoid tyrosine phosphatase or a fragment of lymphoid tyrosine phosphatase including its catalytic domain is determined. In this example the substrate is a synthetic compound, preferably p-NitroPhenyl Phosphate (Rainbow Scientific, Inc, Windsor, Conn. 06095, Cat. No 4400A).

Applicant expresses LYP W620, or LYP R620 in insect cells or a fragment of the enzyme including its catalytic domain in Escherichia coli, or yeast cells. The proteins are expressed with a Glutathione-S-transferase (GST) tag allowing for isolation by affinity chromatography (e.g. vector pEGST, Kholod N, Mustelin T. Novel vectors for co-expression of two proteins in E. coli. Biotechniques. 2001 Aug;31(2):322-3, 326-8). Expressed proteins from the transfected cells are precipitated using Glutathione-Sepharose (Amersham Biosciences Corp, Piscataway, N.J.; Cat. N. 17-5132-02. The precipitated protein can be eluted by using glutathione and separated from the GST tag by using thrombin. The precipitate or the eluted proteins are added to individual wells of a multi-wells plate in the presence or absence of a test compound. The wells having no test compound are useful for determining basal levels of phosphatase activity. In addition, a well having no LYP but having substrate is useful as a negative control. The LYP phosphatase activity of each immunoprecipitate in the presence or absence of a test compound is measured using p-NitroPhenyl Phosphate (pNPP) as substrate. Detection of the relative amounts of phosphatase activity is determined at 405/605 mn (kinetic assay), and the results are compared. The results of this kinetic assay are useful for identifying test compounds as inhibitors and stimulators of LYP phosphatase activity. When full-length proteins are used, they are also useful for discovering test compounds that reduce the phosphatase activity of LYP W620 to more near that of the wild-type LYP R620, and the converse as well.

### PHARMACEUTICAL COMPOSITIONS

Methods of using the compounds and pharmaceutical compositions of the invention are also provided herein. The methods involve both in vitro and in vivo uses of the compounds and pharmaceutical compositions for altering preferred nuclear receptor activity, in a cell type specific fashion.

In certain embodiments, the claimed methods involve the discovery and use of immune system modulating compounds.

Once identified as a modulator using a method of the current invention, an agent can be put in a pharmaceutically acceptable formulation, such as those described in Remington's Pharmaceutical Sciences, 18th ed., Mack Publishing Co., Easton, Pa. (1990), incorporated by reference herein, to generate a pharmaceutical composition useful for specific treatment of diseases and pathological conditions.

Agents identified by the methods taught herein can be administered to a patient either by themselves, or in pharmaceutical compositions where it is mixed with suitable carriers or excipient(s). In treating a patient exhibiting a disorder of interest, a therapeutically effective amount of agent or agents such as these is administered. A therapeutically effective dose refers to that amount of the agent resulting in amelioration of symptoms or a prolongation of survival in a patient.

The agents also can be prepared as pharmaceutically acceptable salts. Examples of pharmaceutically acceptable salts include, but are not limited to acid addition salts such as those containing hydrochloride, sulfate, phosphate, sulfamate, acetate, citrate, lactate, tartrate, methanesulfonate, ethanesulfonate, benzenesulfonate, p-toluenesulfonate, cyclohexylsulfamate and quinate. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid.

Pharmaceutically acceptable salts can be prepared by standard techniques. For example, the free base form of the agent is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipients can be used to facilitate administration of the agent, for example, to increase the solubility of the agent. Examples of carriers and excipients include calcium carbonate, calcium phosphate, various sugars or types of starch, cellulose derivatives, gelatin, vegetable oils, polyethylene glycols and physiologically compatible solvents.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Agents exhibiting large therapeutic indices are preferred. The data obtained from these cell culture assays and animal studies can be used in formulating a range of dosage for use in humans. The dosage of such agents lies preferably within a range of circulating concentrations that include the ED50 with little or no toxicity. The dosage may vary within this range depending upon the dosage form employed and the route of administration utilized.

For any agent identified by the methods taught herein, the therapeutically effective dose can be estimated initially from cell culture assays. For example, a dose can be formulated in animal models to achieve a circulating plasma concentration range that includes the IC50 as determined in cell culture (i.e., the concentration of the test agent which achieves a half-maximal disruption of the protein complex or a half-maximal inhibition of the cellular level and/or activity of a complex component). Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., in The Pharmacological Basis of Therapeutics, Ch. 1 p. 1 (1975)). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the agents herein disclosed into dosages suitable for systemic administration is contemplated. With proper choice of carrier and suitable manufacturing practice, these agents, in particular, those formulated as solutions, may be administered parenterally, such as by intravenous injection. The agents can be formulated readily using pharmaceutically acceptable carriers well known in the art into dosages suitable for oral administration. Such carriers enable the agents of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, and then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the context of the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active agents into preparations which can be used pharmaceutically. The preparations formulated for oral administration may be in the form of tablets, dragees, capsules, or solutions. The pharmaceutical compositions contemplated by the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active agents in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the agents to allow for the preparation of highly concentrated solutions.

Pharmaceutical preparations for oral use can be obtained by combining the active agents with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agent doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added.

Some methods of delivery that may be used include:
a. encapsulation in liposomes,
b. transduction by retroviral vectors,
c. localization to nuclear compartment utilizing nuclear targeting site found on most nuclear proteins,
d. transfection of cells ex vivo with subsequent reimplantation or administration of the transfected cells,
e. a DNA transporter system.

Various modifications and alterations of the invention will become apparent to those skilled in the art without departing from the spirit and scope of the invention, which is defined by the accompanying claims. For example, it should be noted that steps recited in any method claims below do not necessarily need to be performed in the order that they are recited. Those of ordinary skill in the art will recognize variations in performing the steps from the order in which they are recited. For example, in certain embodiments, steps may be performed simultaneously. The accompanying claims should be constructed with these principles in mind.

```
SEQUENCES
Mutant LYP from Genbank Accession No.: NM_015967
      1 agtggctttt tggaggtgtc tcggccatga cacacatttg acatgccctc cctcaaccta
    61 cttatagact atttttcttg ctctgaagca tggaccaaag agaaattctg cagaagttcc
  121 tggatgaggc ccaaagcaag aaaattacta aagaggagtt tgccaatgaa tttctgaagc
  181 tgaaaaggca atctaccaag tacaaggcag acaaaaccta tcctacaact gtggctgaga
  241 agcccaagaa tatcaagaaa aacagatata aggatatttt gccctatgat tatagccggg
  301 tagaactatc cctgataacc tctgatgagg attccagcta catcaatgcc aacttcatta
  361 agggagttta tggacccaag gcttatattg ccacccaggg tcctttatct acaaccctcc
  421 tggacttctg gaggatgatt tgggaatata gtgtccttat cattgttatg gcatgcatgg
  481 agtatgaaat gggaaagaaa aagtgtgagc gctactgggc tgagccagga gagatgcagc
  541 tggaatttgg ccctttctct gtatcctgtg aagctgaaaa aaggaaatct gattatataa
  601 tcaggactct aaaagttaag ttcaatagtg aaaatcgaac tatctaccag tttcattaca
  661 agaattggcc agaccatgat gtaccttcat ctatagaccc tattcttgag ctcatctggg
  721 atgtacgttg ttaccaagag gatgacagtg ttcccatatg cattcactgc agtgctggct
  781 gtggaaggac tggtgttatt tgtgctattg attatacatg gatgttgcta aaagatggga
  841 taattcctga gaacttcagt gttttcagtt tgatccggga aatgcggaca cagaggcctt
  901 cattagttca aacgcaggaa caatatgaac tggtctacaa tgctgtatta gaactattta
  961 agagacagat ggatgttatc agagataaac attctggaac agagagtcaa gcaaagcatt
1021 gtattcctga gaaaaatcac actctccaag cagactctta ttctcctaat ttaccaaaaa
1081 gtaccacaaa agcagcaaaa atgatgaacc aacaaaggac aaaaatggaa atcaaagaat
1141 cttcttcctt tgactttagg acttctgaaa taagtgcaaa agaagagcta gttttgcacc
1201 ctgctaaatc aagcacttct tttgactttc tggagctaaa ttacagtttt gacaaaaatg
1261 ctgacacaac catgaaatgg cagacaaagg catttccaat agttggggag cctcttcaga
1321 agcatcaaag tttggatttg ggctctcttt tgtttgaggg atgttctaat tctaaacctg
1381 taaatgcagc aggaagatat tttaattcaa aggtgccaat aacacggacc aaatcaactc
1441 cttttgaatt gatacagcag agagaaacca aggaggtgga cagcaaggaa aacttttctt
1501 atttggaatc tcaaccacat gattcttgtt ttgtagagat gcaggctcaa aaagtaatgc
1561 atgtttcttc agcagaactg aattattcac tgccatatga ctctaaacac caaatacgta
1621 atgcctctaa tgtaaagcac catgactcta gtgctcttgg tgtatattct tacatacctt
1681 tagtggaaaa tccttatttt tcatcatggc ctccaagtgg taccagttct aagatgtctc
1741 ttgatttacc tgagaagcaa gatggaactg tttttccttc ttctctgttg ccaacatcct
1801 ctacatccct cttctcttat tacaattcac atgattcttt atcactgaat tctccaacca
1861 atatttcctc actattgaac caggagtcag ctgtactagc aactgctcca aggatagatg
1921 atgaaatccc ccctccactt catgtatgga cacctgaatc atttattgtg gttgaggaag
1981 ctggagaatt ctcaccaaat gttcccaaat ccttatccta agctgtgaag gtaaaaattg
2041 gaacatcact ggaatggggt ggaacatctg aaccaaagaa atttgatgac tctgtgatac
2101 ttagaccaag caagagtgta aaactccgaa gtcctaaatc agaactacat caagatcgtt
2161 cttctccccc acctcctctc ccagaaagaa ctctagagtc cttctttctt gccgatgaag
2221 attgtatgca ggcccaatct atagaaacat attctactag ctatcctgac accatggaaa
2281 attcaacatc ttcaaaacag acactgaaga ctcctggaaa aagtttcaca aggagtaaga
2341 gtttgaaaat tttgcgaaac atgaaaaaga gtatctgtaa ttcttgcaca ccaaacaagc
2401 ctgcagaatc tgttcagtca aataactcca gctcatttct gaattttggt tttgcaaacc
2461 gtttttcaaa acccaaagga ccaaggaatc caccaccaac ttggaatatt taataaaact
2521 ccagatttat aataatatgg gctgcaagta cacctgcaaa taaaactact agaatactgc
2581 tagttaaaat aagtgctcta tatgcataat atcaaatatg aagatatgct aatgtgttaa
2641 tagcttttaa aagaaaagca aaatgccaat aagtgccagt tttgcatttt catatcattt
2701 gcattgagtt gaaaactgca aataaaagtt tgtcacttga gcttatgtac agaatgctat
2761 atgagaaaca cttttagaat ggatttattt ttcatttttg ccagttattt ttattttctt
2821 ttacttttct acataaacat aaacttcaaa aggtttgtaa gatttggatc tcaactaatt
2881 tctacattgc cagaatatac tataaaaagt taaaaaaaaa acttactttg tgggttgcaa
2941 tacaaactgc tcttgacaat gactattccc tgacagttat ttttgcataa atggagtata
3001 ccttgtaaat cttcccaaat gttgtggaaa actggaatat taagaaaatg agaaattata
3061 tttattagaa taaaatgtgc aaataatgac aattatttga atgtaacaag gaattcaact
3121 gaaatcctga taagttttaa ccaaagtcat taaattacca attctagaaa agtaatcaat
3181 gaaatataat agctatcttt tggtagcaaa agatataaat tgtatatgtt tatacaggat
3241 ctttcagatc atgtgcaatt tttatctaac caatcagaaa tactagttta aaatgaattt
3301 ctatatgaat atggatctgc cataagaaaa tctagttcaa ctctaatttt atgtagtaaa
3361 taaattggca ggtaattgtt tttacaaaga atccacctga cttcccctaa tgcattaaaa
3421 atatttttat ttaaataact ttatttataa cttttagaaa catgtagtat tgtttaaaca
3481 tcatttgttc ttcagtattt ttcatttgga agtccaatag ggcaaattga atgaagtatt
3541 attatatgtc tcttgtagta caatgtatcc aacagacact caataaactt tttggttgtt
3601 aaaaaaaaaa aaaaa
```

Wild Type LYP, nucleotide 1947 in Genbank Accession No.: NM_{—}015967 is changed from a c to a g.

P1 Domain is nucleotides 1903 to 2034 in Genbank Accession No.: NM_{—}015967 and the wild type has a c nucleotide at position 1947, while the mutant has a g nucleotide at position 1947.

Construct 1 and construct 2, discussed above, are nucleotides 1897-2220 of Genbank Accession No.: NM_{—}015967. Construct 1 has a c at position 1947 while construct 2 gas a g at position 1947.

The SH3 domain of Csk (amino acids 1-55) is nucleotides 413 to 578 of Genbank Accession No.: NM_{—}004383.

```
      1 tccggggcgg cccccggcag ccagcgcgac gttccaaaat cgaacctcag tggcggcgct
    61 cggaagcgga actctgccgg ggccgcgccg gctacattgt ttcctccccc cgactccctc
  121 ccgccccctt cccccgcctt tcttccctcc gcgacccggg ccgtgcgtac gtccccctgc
  181 ctctgcctgg cggtccctcc tcccctctcc ttgcacccat acctctttgt accgcacccc
  241 ctggggaccc ctgcgcccct cccctccccc ctgaccgcat ggaccgtccc gcaggccgct
  301 gatgccgccc gcggcgaggt ggcccggacc gcagtgcccc aagagagctc taatggtacc
  361 aagtgacagg ttggctttac tgtgactcgg ggacgccaga gatcctgaga agatgtcagc
  421 aatacaggcc gcctggccat ccggtacaga atgtattgcc aagtacaact tccacggcac
  481 tgccgagcag gacctgccct tctgcaaagg agacgtgctc accattgtgg ccgtcaccaa
  541 ggaccccaac tggtacaaag ccaaaaacaa ggtgggccgt gagggcatca tcccagccaa
  601 ctacgtccag aagcgggagg gcgtgaaggc gggtaccaaa ctcagcctca tgccttggtt
  661 ccacggcaag atcacacggg agcaggctga gcggcttctg tacccgccgg agacaggcct
  721 gttcctggtg cgggagagca ccaactaccc aggagactac acgctgtgcg tgagctgcga
  781 cggcaaggtg gagcactacc gcatcatgta ccatgccagc aagctcagca tcgacgagga
  841 ggtgtacttt gagaacctca tgcagctggt ggagcactac acctcagacg cagatggact
  901 ctgtacgcgc ctcattaaac caaaggtcat ggagggcaca gtggcggccc aggatgagtt
  961 ctaccgcagc ggctgggccc tgaacatgaa ggagctgaag ctgctgcaga ccatcgggaa
1021 gggggagttc ggagacgtga tgctgggcga ttaccgaggg aacaaagtcg ccgtcaagtg
1081 cattaagaac gacgccactg cccaggcctt cctggctgaa gcctcagtca tgacgcaact
1141 gcggcatagc aacctggtgc agctcctggg cgtgatcgtg gaggagaagg gcgggctcta
1201 catcgtcact gagtacatgg ccaaggggag ccttgtggac tacctgcggt ctaggggtcg
1261 gtcagtgctg ggcggagact gtctcctcaa gttctcgcta gatgtctgcg aggccatgga
1321 atacctggag ggcaacaatt tcgtgcatcg agacctggct gcccgcaatg tgctggtgtc
1381 tgaggacaac gtggccaagg tcagcgactt tggtctcacc aaggaggcgt ccagcaccca
1441 ggacacgggc aagctgccag tcaagtggac agcccctgag gccctgagag agaagaaatt
1501 ctccactaag tctgacgtgt ggagtttcgg aatccttctc tgggaaatct actcctttgg
1561 gcgagtgcct tatccaagaa ttcccctgaa ggacgtcgtc cctcgggtgg agaagggcta
1621 caagatggat gcccccgacg gctgcccgcc cgcagtctat gaagtcatga agaactgctg
1681 gcacctggac gccgccatgc ggccctcctt cctacagctc cgagagcagc ttgagcacat
1741 caaaacccac gagctgcacc tgtgacggct ggcctccgcc tgggtcatgg gcctgtgggg
1801 actgaacctg gaagatcatg gacctggtgc ccctgctcac tgggcccgag cctgaactga
1861 gccccagcgg gctggcgggc ctttttcctg cgtcccagcc tgcaccactc cggccccgtc
1921 tctcttggac ccacctgtgg ggcctgggga gcccactgag gggccaggga ggaaggaggc
1981 cacggagcgg gcggcagcgc cccaccacgt cgggcttccc tggcctcccg ccactcgcct
2041 tcttagagtt ttattccttt ccttttttga gatttttttt ccgtgtgttt attttttatt
2101 atttttcaag ataaggagaa agaaagtacc cagcaaatgg gcattttaca agaagtacga
2161 atcttatttt tcctgtcctg cccgtgaggt gggggggacc gggcccctct ctagggaccc
2221 ctcgccccag cctcattccc cattctgtgt cccatgtccc gtgtctcctc ggtcgccccg
2281 tgtttgcgct tgaccatgtt gcactgtttg catgcgcccg aggcagacgt ctgtcagggg
2341 cttggatttc gtgtgccgct gccacccgcc cacccgcctt gtgagatgga atcgtaataa
2401 accacgccat gaggaaaaaa
```

We claim:

1. A diagnostic screening method to determine the presence of in vivo characteristics leading to an autoimmune disorder in a mammal comprising the steps of:
a. isolating an in vivo component from a person to be screened for a characteristic leading to an autoimmune disorder;
b. performing a screening assay using said isolated in vivo component;
c. comparing a result derived from the screening assay with a known value for that characteristic;
d. correlating said in vivo characteristic to a known characteristic leading to an autoimmune disorder, such that the presence of at least one characteristic indicates an individual's susceptibility to an autoimmune disorder stemming from dysregulation of lymphoid tyrosine phosphatase mediated T-cell activation.

2. The diagnostic screening method of claim 1, wherein the in vivo component is nucleic acid.

3. The diagnostic screening method of claim 2, wherein the in vivo component is genomic DNA.

4. The diagnostic screening method of claim 2, wherein the in vivo component is the mammalian chromosome 1p13.

5. The diagnostic screening method of claim 2, wherein the in vivo component is the PTPN22 gene.

6. The diagnostic screening method of claim 2, wherein the in vivo component is nucleotides 1858-1860 of the PTPN22 gene.

7. The diagnostic screening method of claim 2, wherein the in vivo component is nucleotide 1858 of the PTPN22 gene.

8. The diagnostic screening method of claim 1, wherein the in vivo component is a cell.

9. The diagnostic screening method of claim 8, wherein the in vivo component is a lymphocyte.

10. The diagnostic screening method of claim 1, wherein the in vivo component is a phosphatase.

11. The diagnostic screening method of claim 10, wherein the in vivo component is lymphoid tyrosine phosphatase.

12. The diagnostic screening method of claim 1, wherein the in vivo component is one or more amino acids included in the sequence of a phosphatase peptide.

13. The diagnostic screening method of claim 1, wherein the in vivo component is the c-terminal fragment of a lymphoid tyrosine phosphatase.

14. The diagnostic screening method of claim 1, wherein the in vivo component is residue 620 of a lymphoid tyrosine phosphatase.

15. The diagnostic screening method of claim 1, wherein the autoimmune disorder being screened for is a disorder selected from the group consisting of type-1 diabetes, systemic lupus erythematosus, rheumatoid arthritis, juvenile arthritis, Graves disease and Addison disease.

16. The diagnostic screening method of claim 1, wherein the screening assay is a genotyping assay.

17. The diagnostic screening method of claim 16, wherein the genotyping assay is an assay selected from the group consisting of restriction fragment length polymorphism, DNA sequencing, RNA sequencing, direct sequencing, single-stranded conformational polymorphism, heteroduplex analysis, primer extension, oligonucleotide ligation, allele specific digestion, flap endonuclease discrimination and allele specific hybridization.

18. The diagnostic screening method of claim 17, wherein the genotyping assay determines the nucleotide present at position 1858 in the PTPN22 gene.

19. The diagnostic screening method of claim 1, wherein the screening assay is a phenotyping assay.

20. The diagnostic screening method of claim 19, wherein the phenotyping assay is an assay selected from the group consisting of protein sequencing, signal transduction cascades, binding assays, western blots, phosphatase activity assay and pull-down assay.

21. The diagnostic screening method of claim 20, wherein the phenotyping assay determines the amino acid residue present at codon 620 of the Lymphoid Tyrosine Phosphatase protein.

22. A method of screening for agents that modulate lymphoid tyrosine phosphatase comprising the steps of:
a. providing a system further comprising;
(i) a peptide similar to Csk that can specifically interact with a peptide similar to lymphoid tyrosine phosphatase;
(ii) at least one peptide similar to lymphoid tyrosine phosphatase and at least comprising an amino acid that is situated substantially similarly with respect to its tertiary structure as the amino acid at position 620 in wild-type lymphoid tyrosine phosphatase protein is situated; and
(iii) a reporter system to report the interaction of the peptide similar to Csk with the peptide similar to lymphoid tyrosine phosphatase;
b. introducing a test compound to the system;
c. determining the effect that the test compound has on the system;
d. correlating the test compound with the effect on the system; and
e. identifying the correlated test compound as an agent that modulates lymphoid tyrosine phosphatase.

23. The method of screening for agents that modulate lymphoid tyrosine phosphatase of claim 22, wherein the system provided is a system selected from the group consisting of cell lines, recombinant cell lines, expression systems, baculovirus systems, extracted proteins, affinity columns, computer algorithms and multi-well plates.

24. The method of screening for agents that modulate lymphoid tyrosine phosphatase of claim 22, wherein the peptide similar to Csk is endogenous Csk protein.

25. The method of screening for agents that modulate lymphoid tyrosine phosphatase of claim 22, wherein the peptide similar to Csk is exogenous Csk protein.

26. The method of screening for agents that modulate lymphoid tyrosine phosphatase of claim 22, wherein the peptide similar to Csk is a fragment of Csk at least comprising the SH3 domain.

27. The method of screening for agents that modulate lymphoid tyrosine phosphatase of claim 22, wherein the peptide similar to lymphoid tyrosine phosphatase is endogenous lymphoid tyrosine phosphatase.

28. The method of screening for agents that modulate lymphoid tyrosine phosphatase n of claim 22, wherein the peptide similar to lymphoid tyrosine phosphatase is exogenous lymphoid tyrosine phosphatase.

29. The method of screening for agents that modulate lymphoid tyrosine phosphatase of claim 22, wherein the peptide similar to lymphoid tyrosine phosphatase is a fragment of lymphoid tyrosine phosphatase at least comprising amino acid 620.

30. The method of screening for agents that modulate lymphoid tyrosine phosphatase of claim 22, wherein the system comprises two copies of the peptide similar to lymphoid tyrosine phosphatase.

31. The method of screening for agents that modulate lymphoid tyrosine phosphatase of claim 30, wherein the two copies of the peptide similar to lymphoid tyrosine phosphatase both comprise arginine for the amino acid residue that interacts with amino acid residue W47 of Csk, typically residue 620 of lymphoid tyrosine phosphatase.

32. The method of screening for agents that modulate lymphoid tyrosine phosphatase of claim 30, wherein the two copies of the peptide similar to lymphoid tyrosine phosphatase both comprise tryptophan for the amino acid residue that interacts with amino acid residue W47 of Csk, typically residue 620 of lymphoid tyrosine phosphatase.

33. The method of screening for agents that modulate lymphoid tyrosine phosphatase of claim 30, wherein the two copies of the peptide similar to lymphoid tyrosine phosphatase comprise a first peptide with arginine for the amino acid residue that interacts with amino acid residue W47 of Csk, typically residue 620 of lymphoid tyrosine phosphatase and a second peptide with tryptophan for the amino acid residue that interacts with amino acid residue W47 of Csk, typically residue 620 of lymphoid tyrosine phosphatase.

34. The method of screening for agents that modulate lymphoid tyrosine phosphatase of claim 30, wherein the two copies of the peptide similar to lymphoid tyrosine phosphatase comprise a polymorphism near the amino acid residue 620 causing a structural change in lymphoid tyrosine phosphatase such that the 620 residue is unable to properly contact the ligand binding cleft of the Csk SH3 domain.

35. The method of screening for agents that modulate lymphoid tyrosine phosphatase of claim 30, wherein the one copy of the peptide similar to lymphoid tyrosine phosphatase comprises a polymorphism near the amino acid residue 620 causing a structural change in lymphoid tyrosine phosphatase such that the 620 residue is unable to properly contact the ligand binding cleft of the Csk SH3 domain.

36. The method of screening for agents that modulate lymphoid tyrosine phosphatase of claim 22, wherein the lymphoid tyrosine phosphatase has a shifted amino acid sequence configuration caused by sequence variations selected from the group consisting of splice variants, truncations, additions and fusion proteins.

37. The method of screening for agents that modulate lymphoid tyrosine phosphatase of claim 22, wherein the reporter system is a system selected from the group consisting of two-stage antibody detection, electrophoresis, reporter construct vectors, ELISA, affinity columns, phosphatase activity assay and T-cell activation assay.

38. The method of screening for agents that modulate lymphoid tyrosine phosphatase regulation of claim 22, wherein the test compound is selected from the group consisting of a peptide, peptidomimetic, non-peptidyl compound, carbohydrate, lipid, a synthetic compound, a natural product, an antibody or antibody fragment, a small organic molecules, a small inorganic molecule, and a nucleotide sequence.

39. The method of screening for agents that modulate lymphoid tyrosine phosphatase of claim 22, wherein the step of determining the effect a test compound has on the system includes comparison of the system in the presence of the test compound with the system in the absence of the test compound.

40. An agent that modulates lymphoid tyrosine phosphatase mediated T-cell regulation identified using the method of claim 22.

41. A method for the treatment of an autoimmune disease comprising the steps of administering to a patient diagnosed with an autoimmune disease an agent that modulates the consequences of dysfunctional lymphoid tyrosine phosphatase protein on the lymphoid tyrosine phosphatase mediated T-cell regulation in a quantity sufficient to supplement t-cell regulation to treat autoimmune disease.

42. The treatment method of claim 41, wherein the agent that modulates the consequences of dysfunctional lymphoid tyrosine phosphatase protein is an agent selected from the group consisting of a peptide, peptidomimetic, non-peptidyl compound, carbohydrate, lipid, a synthetic compound, a natural product, an antibody or antibody fragment, a small organic molecules, a small inorganic molecule, and a nucleotide sequence.

43. A method for the treatment of an autoimmune disease comprising the steps of administering to a patient diagnosed with an autoimmune disease an exogenous nucleic acid molecule that modulates the consequences of dysfunctional lymphoid tyrosine phosphatase protein on lymphoid tyrosine phosphatase mediated T-cell regulation in a quantity sufficient to supplement T-cell regulation to treat autoimmune disease.

44. The treatment method of claim 43, wherein the exogenous nucleic acid molecule is administered to a patient using an administration system comprising a nucleic acid vector system, microinjection, a gene gun or a liposome.

45. The treatment method of claim 43, wherein the exogenous nucleic acid molecule is an RNA molecule that has a sequence that is antisense to a portion of the mutant lymphoid tyrosine phosphatase RNA transcript.

46. The treatment method of claim 45, wherein the antisense RNA molecule is specific and sensitive for the mutant lymphoid tyrosine phosphatase RNA transcript but not the wild-type lymphoid tyrosine phosphatase RNA transcript.

47. The treatment method of claim 43, wherein the exogenous nucleic acid molecule is a DNA molecule that provides a sequence coding for the wild-type lymphoid tyrosine phosphatase allele.

48. The treatment method of claim 43, wherein the exogenous nucleic acid molecule supplements for an intronic polymorphism of the lymphoid tyrosine phosphatase allele affecting the expression of wild-type lymphoid tyrosine phosphatase.

49. A substantially purified lymphoid tyrosine phosphatase having increased phosphatase activity.

50. A method for screening for compounds having the activity of inhibiting the increased phosphatase activity of a mutant lymphoid tyrosine phosphatase comprising the steps of:
(a) contacting a substantially purified mutant lymphoid tyrosine phosphatase enzyme having increased phosphatase activity with a compound that may inhibit the phosphatase activity of the substantially purified mutant lymphoid tyrosine phosphatase; and
(b) comparing the phosphatase activity of the substantially purified mutant lymphoid tyrosine phosphatase in the presence and in the absence of the compound, in order to determine whether or not the compound inhibits the phosphatase activity of the substantially purified mutant lymphoid tyrosine phosphatase activity.

51. A substantially purified peptide similar to lymphoid tyrosine phosphatase (LYP)comprising a bulky amino acid at amino acid position 620, wherein the bulky amino acid affects the interaction of LYP and Csk.

52. The substantially purified peptide similar to LYP of claim 51 wherein the bulky amino acid is tyrosine.

53. A nucleic acids that codes for the substantially purified peptide similar to lymphoid tyrosine phosphatase of claim 51.


SEQUENCE LISTING (9 sequences)

SEQ ID NO: 1 - DNA, 3 bp, Homo sapiens
cgg

SEQ ID NO: 2 - DNA, 3 bp, Homo sapiens
tgg

SEQ ID NO: 3 - DNA, 21 bp, Artificial
  Synthetically generated sequence
tcaccagcttcctcaaccaca

SEQ ID NO: 4 - DNA, 25 bp, Artificial
  Synthetically generated sequence
gataatgttgcttcaacggaattta

SEQ ID NO: 5 - DNA, 3615 bp, Homo sapiens
agtggctttttggaggtgtctcggccatgacacacatttgacatgccctccctcaacctacttatagactatttttcttgctctgcagcatggaccaaagagaaattctgcagaagttcctggatgaggcccaaagcaagaaaattactaaagaggagtttgccaatgaatttctgaagctgaaaaggcaatctaccaagtacaaggcagacaaaacctatcctacaactgtggctgagaagcccaagaatatcaagaaaaacagatataaggatattttgccctatgattatagccgggtagaactatccctgataacctctgatgaggattccagctacatcaatgccaacttcattaagggagtttatggacccaaggcttatattgccacccagggtcctttatctacaaccctcctggacttctggaggatgatttgggaatatagtgtccttatcattgttatggcatgcatggagtatgaaatgggaaagaaaaagtgtgagcgctactgggctgagccaggagagatgcagctggaatttggccctttctctgtatcctgtgaagctgaaaaaaggaaatctgattatataatcaggactctaaaagttaagttcaatagtgaaactcgaactatctaccagtttcattacaagaattggccagaccatgatgtaccttcatctatagaccctattcttgagctcatctgggatgtacgttgttaccaagaggatgacagtgttcccatatgcattcactgcagtgctggctgtggaaggactggtgttatttgtgctattgattatacatggatgttgctaaaagatgggataattcctgagaacttcagtgttttcagtttgatccgggaaatgcggacacagaggccttcattagttcaaacgcaggaacaatatgaactggtctacaatgctgtattagaactatttaagagacagatggatgttatcagagataaacattctggaacagagagtcaagcaaagcattgtattcctgagaaaaatcacactctccaagcagactcttattctcctaatttaccaaaaagtaccacaaaagcagcaaaaatgatgaaccaacaaaggacaaaaatggaaatcaaagaatcttcttcctttgactttaggacttctgaaataagtgcaaaagaagagctagttttgcaccctgctaaatcaagcacttcttttgactttctggagctaaattacagttttgacaaaaatgctgacacaaccatgaaatggcagacaaaggcatttccaatagttggggagcctcttcagaagcatcaaagtttggatttgggctctcttttgtttgagggatgttctaattctaaacctgtaaatgcagcaggaagatattttaattcaaaggtgccaataacacggaccaaatcaactccttttgaattgatacagcagagagaaaccaaggaggtggacagcaaggaaaacttttcttatttggaatctcaaccacatgattcttgttttgtagagatgcaggctcaaaaagtaatgcatgtttcttcagcagaactgaattattcactgccatatgactctaaacaccaaatacgtaatgcctctaatgtaaagcaccatgactctagtgctcttggtgtatattcttacatacctttagtggaaaatccttatttttcatcatggcctccaagtggtaccagttctaagatgtctcttgatttacctgagaagcaagatggaactgtttttccttcttctctgttgccaacatcctctacatccctcttctcttattacaattcacatgattctttatcactgaattctccaaccaatatttcctcactattgaaccaggagtcagctgtactagcaactgctccaaggatagatgatgaaatcccccctccacttcctgtatggacacctgaatcatttattgtggttgaggaagctggagaattctcaccaaatgttcccaaatccttatcctcagctgtgaaggtaaaaattggaacatcactggaatggggtggaacatctgaaccaaagaaatttgatgactctgtgatacttagaccaagcaagagtgtaaaactccgaagtcctaaatcagaactacatcaagatcgttcttctcccccacctcctctcccagaaagaactctagagtccttctttcttgccgatgaagattgtatgcaggcccaatctatagaaacatattctactagctatcctgacaccatggaaaattcaacatcttcaaaacagacactgaagactcctggaaaaagtttcacaaggagtaagagtttgaaaattttgcgaaacatgaaaaagagtatctgtaattcttgcccaccaaacaagcctgcagaatctgttcagtcaaataactccagctcatttctgaattttggttttgcaaaccgtttttcaaaacccaaaggaccaaggaatccaccaccaacttggaatatttaataaaactccagatttataataatatgggctgcaagtacacctgcaaataaaactactagaatactgctagttaaaataagtgctctatatgcataatatcaaatatgaagatatgctaatgtgttaatagcttttaaaagaaaagcaaaatgccaataagtgccagttttgcattttcatatcatttgcattgagttgaaaactgcaaataaaagtttgtcacttgagcttatgtacagaatgctatatgagaaacacttttagaatggatttatttttcatttttgccagttatttttattttcttttacttttctacataaacataaacttcaaaaggtttgtaagatttggatctcaactaatttctacattgccagaatatactataaaaagttaaaaaaaaaacttactttgtgggttgcaatacaaactgctcttgacaatgactattccctgacagttatttttgcctaaatggagtataccttgtaaatcttcccaaatgttgtggaaaactggaatattaagaaaatgagaaattatatttattagaataaaatgtgcaaataatgacaattatttgaatgtaacaaggaattcaactgaaatcctgataagttttaaccaaagtcattaaattaccaattctagaaaagtaatcaatgaaatataatagctatcttttggtagcaaaagatataaattgtatatgtttatacaggatctttcagatcatgtgcaatttttatctaaccaatcagaaatactagtttaaaatgaatttctatatgaatatggatctgccataagaaaatctagttcaactctaattttatgtagtaaataaattggcaggtaattgtttttacaaagaatccacctgacttcccctaatgcattaaaaatatttttatttaaataactttatttataacttttagaaacatgtagtattgtttaaacatcatttgttcttcagtatttttcatttggaagtccaatagggcaaattgaatgaagtattattatctgtctcttgtagtacaatgtatccaacagacactcaataaactttttggttgttaaaaaaaaaaaaaaa

SEQ ID NO: 6 - DNA, 2420 bp, Homo sapiens
tccggggcggcccccggcagccagcgcgacgttccaaaatcgaacctcagtggcggcgctcggaagcggaactctgccggggccgcgccggctacattgtttcctccccccgactccctcccgcccccttcccccgcctttcttccctccgcgacccgggccgtgcgtccgtccccctgcctctgcctggcggtccctcctcccctctccttgcacccatacctctttgtaccgcaccccctggggacccctgcgcccctcccctcccccctgaccgcatggaccgtcccgcaggccgctgatgccgcccgcggcgaggtggcccggaccgcagtgccccaagagagctctaatggtaccaagtgacaggttggctttactgtgactcggggacgccagagctcctgagaagatgtcagcaatacaggccgcctggccatccggtacagaatgtattgccaagtacaacttccacggcactgccgagcaggacctgcccttctgcaaaggagacgtgctcaccattgtggccgtcaccaaggaccccaactggtacaaagccaaaaacaaggtgggccgtgagggcatcatcccagccaactacgtccagaagcgggagggcgtgaaggcgggtaccaaactcagcctcatgccttggttccacggcaagatcacacgggagcaggctgagcggcttctgtacccgccggagacaggcctgttcctggtgcgggagagcaccaactaccccggagactacacgctgtgcgtgagctgcgacggcaaggtggagcactaccgcatcatgtaccatgccagcaagctcagcatcgacgaggaggtgtactttgagaacctcatgcagctggtggagcactacacctcagacgcagatggactctgtacgcgcctcattaaaccaaaggtcatggagggcacagtggcggcccaggatgagttctaccgcagcggctgggccctgaacatgaaggagctgaagctgctgcagaccatcgggaagggggagttcggagacgtgatgctgggcgattaccgagggaacaaagtcgccgtcaagtgcattaagaacgacgccactgcccaggccttcctggctgaagcctcagtcatgacgcaactgcggcatagcaacctggtgcagctcctgggcgtgatcgtggaggagaagggcgggctctacatcgtcactgagtacatggccaaggggagccttgtggactacctgcggtctaggggtcggtcagtgctgggcggagactgtctcctcaagttctcgctagatgtctgcgaggccatggaatacctggagggcaacaatttcgtgcatcgagacctggctgcccgcaatgtgctggtgtctgaggacaacgtggccaaggtcagcgactttggtctcaccaaggaggcgtccagcacccaggacacgggcaagctgccagtcaagtggacagcccctgaggccctgagagagaagaaattctccactaagtctgacgtgtggagtttcggaatccttctctgggaaatctactcctttgggcgagtgccttatccaagaattcccctgaaggacgtcgtccctcgggtggagaagggctacaagatggatgcccccgacggctgcccgcccgcagtctatgaagtcatgaagaactgctggcacctggacgccgccatgcggccctccttcctacagctccgagagcagcttgagcacatcaaaacccacgagctgcacctgtgacggctggcctccgcctgggtcatgggcctgtggggactgaacctggaagatcatggacctggtgcccctgctcactgggcccgagcctgaactgagccccagcgggctggcgggcctttttcctgcgtcccagcctgcacccctccggccccgtctctcttggacccacctgtggggcctggggagcccactgaggggccagggaggaaggaggccacggagcgggcggcagcgccccaccacgtcgggcttccctggcctcccgccactcgccttcttagagttttattcctttccttttttgagattttttttccgtgtgtttattttttattatttttcaagataaggagaaagaaagtacccagcaaatgggcattttacaagaagtacgaatcttatttttcctgtcctgcccgtgaggtgggggggaccgggcccctctctagggacccctcgccccagcctcattccccattctgtgtcccatgtcccgtgtctcctcggtcgccccgtgtttgcgcttgaccatgttgcactgtttgcatgcgcccgaggcagacgtctgtcaggggcttggatttcgtgtgccgctgccacccgcccacccgccttgtgagatggaatcgtaataaaccacgccatgaggaaaaaa

SEQ ID NO: 7 - protein, 23 aa, Homo sapiens
RRTDDEIPPPLPERTPESFIVVE

SEQ ID NO: 8 - protein, 23 aa, Homo sapiens
RRTDDEIPPPLPEWTPESFIVVE

SEQ ID NO: 9 - DNA, 15 bp, Homo sapiens
  misc_feature (7)..(7): n is t or c
cctgtayggacacct